(19) United States
(12) Patent Application Publication
(10) Pub. No.: US 2016/0086393 A1
(43) Pub. Date: Mar. 24, 2016

(54) CUSTOMIZED VEHICLE MONITORING PRIVACY SYSTEM

(71) Applicant: The Travelers Indemnity Company, Hartford, CT (US)

(72) Inventors: Dean M. Collins, Manchester, CT (US); Bryan Smith, Glastonbury, CT (US)

(21) Appl. No.: 14/957,701

(22) Filed: Dec. 3, 2015

Related U.S. Application Data
(63) Continuation-in-part of application No. 13/316,700, filed on Dec. 12, 2011, now abandoned.
Application No. 13/316,700 is a continuation-in-part of application No. 13/109,418, filed on May 17, 2011, now abandoned.
(60) Provisional application No. 61/345,220, filed on May 17, 2010.
(60) Provisional application No. 61/427,201, filed on Dec. 26, 2010.

(51) Int. Cl. G07C 5/00; A61B 5/11; A61B 5/024; A61B 5/08; G07C 5/06; G07C 5/08

(52) U.S. Cl. CPC G07C 5/008; G07C 5/06; G07C 5/0808; A61B 5/024; A61B 5/0816; A61B 5/11

(57) ABSTRACT

Systems and methods for customizable vehicle parameter sensing, recording, and utilization.

### CROSS-REFERENCE TO RELATED APPLICATIONS

This application claims benefit and priority under 35 U.S.C. §120 to, and is a Continuation-In-Part (CIP) of, U.S. patent application Ser. No. 13/316,700 filed on Dec. 12, 2011 in the name of Collins et al. and titled “MONITORING CUSTOMER-SELECTED VEHICLE PARAMETERS IN ACCORDANCE WITH CUSTOMER PREFERENCES”, which itself claims benefit and priority (i) under 35 U.S.C. §120 to, and is a Continuation-In-Part (CIP) of, U.S. patent application Ser. No. 13/109,418 filed on May 17, 2011 in the name of Collins et al. and titled “MONITORING CUSTOMER-SELECTED VEHICLE PARAMETERS”, which itself claims benefit and priority under 35 U.S.C. §119(e) to U.S. Provisional Application No. 61/345,220, filed May 17, 2010, entitled “Systems, Methods, and Media for Determining Insurance Premiums Based on Customer-Selected Data Items,” and (ii) under 35 U.S.C. §119(e) to U.S. Provisional Application No. 61/427,201, filed on Dec. 26, 2010 and titled “RISK ZONES”, each of the above-referenced applications of which is incorporated by reference herein in its entirety.

This application is also related to U.S. patent application Ser. No. 13/316,754 filed on Dec. 12, 2011 in the name of Collins et al. and titled “MONITORING CLIENT-SELECTED VEHICLE PARAMETERS IN ACCORDANCE WITH CLIENT PREFERENCES”, which itself claims priority to U.S. patent application Ser. No. 13/109,449 filed on May 17, 2011 in the name of Collins et al. and titled “MONITORING CUSTOMER-SELECTED VEHICLE PARAMETERS”.

### BACKGROUND

Various types of vehicle monitoring applications and systems are available that enable monitoring and/or analysis of parameters associated with vehicle operations. Conventional systems, however, are implemented in a manner that is perceived to benefit the consumer of the vehicle data and accordingly do not take into account privacy concerns.

### BRIEF DESCRIPTION OF THE DRAWINGS

The accompanying drawings, which are incorporated in and constitute a part of this specification, illustrate exemplary embodiments of the invention and, together with the description, serve to explain the principles of the invention. In the drawings:

FIG. 1 is a diagram of a system according to some embodiments;

FIG. 2 is a diagram of a system according to some embodiments;

FIG. 3 is a diagram of a system according to some embodiments;

FIG. 4A and FIG. 4B are flowcharts of a method according to some embodiments;

FIG. 5 is a flowchart of a method according to some embodiments;

FIG. 6 depicts an example user interface according to some embodiments;

FIG. 7 depicts an example user interface according to some embodiments;

FIG. 8 depicts an example user interface according to some embodiments;

FIG. 9 depicts an example user interface according to some embodiments;

FIG. 10 depicts an example user interface according to some embodiments; and

FIG. 11 depicts an example user interface according to some embodiments.

### DETAILED DESCRIPTION

This disclosure relates to systems, media, and methods for recording customer-selected and/or customer-customized data items relating to vehicular operations (e.g., vehicle monitoring in accordance with customer-selected and/or defined privacy settings). In some embodiments, customer-selected and/or customer-customized data items relating to use of a customer's vehicle or vehicles may be monitored, recorded, and/or transmitted to an end-user or data consumer (e.g., an insurance company), responsive to customer control. The insurance company may determine an insurance premium based on values associated with the customer-selected data items, alone or in conjunction with other data. In addition, the customer may choose, select, and/or update, in real-time, which data items are monitored, recorded, and/or transmitted, and may receive confirmation that the system has been updated based on the customer's preferences. The policy itself may or may not be updated (e.g., policy pricing, discounts, surcharges, and/or associated benefits or penalties) immediately to reflect these updates. The customer may select different data items to be monitored, recorded, and/or transmitted for different individuals, vehicles, classes of vehicles, and/or may indicate the vehicle an individual is operating. Different customers may have different restrictions as to the data items they may select or unselect for monitoring, based, for example, on the type of policy and/or customer type. The customer may be, e.g., an individual, a family, etc.

As used herein, “automobile” and “vehicle” may be used interchangeably and may generally refer to any type, configuration, and/or quantity of vehicles, such as vehicles of the type typically covered by an automobile insurance policy, a recreational vehicle insurance policy, a boat insurance policy, and other related policies.

As utilized herein, the term “customer” may generally refer to any type, quantity, and or manner of entity with or for which policy, telematics, and/or premium information may be determined in accordance with embodiments described herein. A customer may comprise an individual or personal insurance policy holder, for example, and/or may comprise an individual, family, and/or other entity that seeks to price and/or obtain an insurance and/or other underwriting policy as described herein. A customer may have an existing business relationship with other entities described herein, such as an insurance company for example, or may not yet have such a relationship—i.e., a “customer” may comprise a “potential customer”. Moreover, a customer may be a “user” of one or more of the systems described herein.

As used herein, the term “network component” may refer to a user or network device, or a component, piece, portion, or combination of user or network devices. Examples of network components may include a Static Random Access Memory (SRAM) device or module, a network processor, and a network communication path, connection, port, or cable.

In addition, some embodiments are associated with a “network” or a “communication network.” As used herein, the terms “network” and “communication network” may be used interchangeably and may refer to any object, entity, component, device, and/or any combination thereof that permits, facilitates, and/or otherwise contributes to or is associated with the transmission of messages, packets, signals, and/or other forms of information between and/or within one or more network devices. Networks may be or include a plurality of interconnected network devices. In some embodiments, networks may be hard-wired, wireless, virtual, neural, and/or any other configuration or type that is or becomes known. Communication networks may include, for example, devices that communicate directly or indirectly, via a wired or wireless medium such as the Internet, intranet, LAN, WAN, Ethernet (or IEEE 802.3), Token Ring, or via any appropriate communications means or combination of communications means. Exemplary protocols include but are not limited to: Bluetooth™, Time Division Multiple Access (TDMA), Code Division Multiple Access (CDMA), Global System for Mobile communications (GSM), Enhanced Data rates for GSM Evolution (EDGE), General Packet Radio Service (GPRS), Wideband CDMA (WCDMA), Advanced Mobile Phone System (AMPS), Digital AMPS (D-AMPS), IEEE 802.11 (WI-FI), IEEE 802.3, SAP, the best of breed (BOB), and/or system to system (S2S).

In cases where video signals or large files are being sent over the network, a broadband network may be used to alleviate delays associated with the transfer of such large files, however, such an arrangement is not required. Each of the devices may be adapted to communicate on such a communication means. Any number and type of machines may be in communication via the network. Where the network is the Internet, communications over the Internet may be through a website maintained by a computer on a remote server or over an online data network, including commercial online service providers, and/or bulletin board systems. In yet other embodiments, the devices may communicate with one another over RF, cable TV, and/or satellite links. Where appropriate, encryption or other security measures, such as logins and passwords, may be provided to protect proprietary or confidential information.

As used herein, the terms “information” and “data” may be used interchangeably and may refer to any data, text, voice, video, image, message, bit, packet, pulse, tone, waveform, and/or other type or configuration of signal and/or information. Information may comprise information packets transmitted, for example, in accordance with the Internet Protocol Version 6 (IPv6) standard. Information may, according to some embodiments, be compressed, encoded, encrypted, and/or otherwise packaged or manipulated in accordance with any method that is or becomes known or practicable.

As used herein, “determining” includes calculating, computing, deriving, looking up (e.g., in a table, database, or data structure), ascertaining, and/or recognizing.

A “processor” means any one or more microprocessors, Central Processing Unit (CPU) devices, computing devices, microcontrollers, and/or digital signal processors. Exemplary processors include the INTEL PENTIUM and AMD ATHLON processors.

The terms “computer-readable medium” and “computer-readable memory” refer to any medium that participates in providing data (e.g., instructions) that may be read by a computer and/or a processor. Such a medium may take many forms, including but not limited to non-volatile media, volatile media, and other specific types of transmission media. Non-volatile media include, for example, optical or magnetic disks and other persistent memory. Volatile media include DRAM, which typically constitutes the main memory. Other types of transmission media include coaxial cables, copper wire, and fiber optics, including the wires that comprise a system bus coupled to the processor.

Common forms of computer-readable media include, for example, a floppy disk, a flexible disk, hard disk, magnetic tape, any other magnetic medium, a CD-ROM, Digital Video Disc (DVD), any other optical medium, punch cards, paper tape, any other physical medium with patterns of holes, a RAM, a PROM, an EPROM, a FLASH-EEPROM, a USB memory stick, a dongle, any other memory chip or cartridge, a carrier wave, or any other medium from which a computer can read. The terms “computer-readable medium” and/or “tangible media” specifically exclude signals, waves, and wave forms or other intangible or transitory media that may nevertheless be readable by a computer.

Various forms of computer-readable media may be involved in carrying sequences of instructions to a processor. For example, sequences of instruction (i) may be delivered from RAM to a processor, (ii) may be carried over a wireless transmission medium, and/or (iii) may be formatted according to numerous formats, standards or protocols. For a more exhaustive list of protocols, the term “network” is defined above and includes many exemplary protocols that are also applicable here.

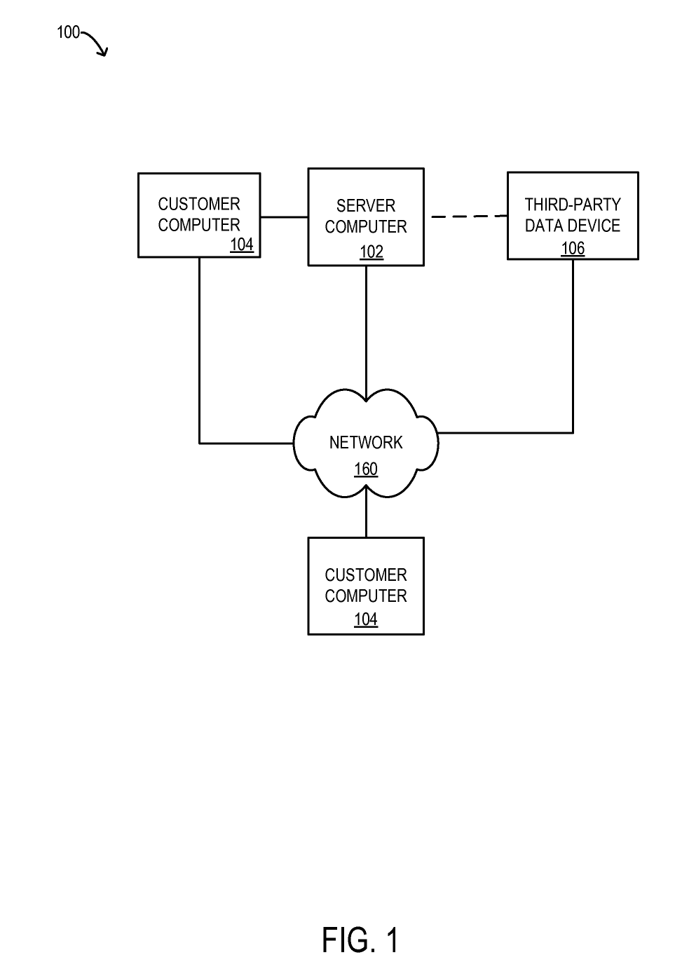

FIG. 1 depicts a block diagram of an example system 100 according to some embodiments. The system 100 may comprise one or more customer computers 104 (e.g., operated by one or more customers; not explicitly shown in FIG. 1) in communication with a server computer 102 (e.g., a controller device) via a network 160. Typically a processor (e.g., one or more microprocessors, one or more microcontrollers, one or more digital signal processors) of a customer computer 104 or server computer 102 will receive instructions (e.g., from a memory device), and execute those instructions, thereby performing one or more processes defined by those instructions. Instructions may be embodied in, for example, one or more computer programs and/or one or more scripts.

In some embodiments a server computer 102 and/or one or more of the customer computers 104 stores and/or has access to data items related to insurance business information. Such information may include insurance data, such as policy data and underwriting rules, for example, and customer data, such as demographic data and data relating to use of one or more of the customer's vehicles (not shown in FIG. 1), for example.

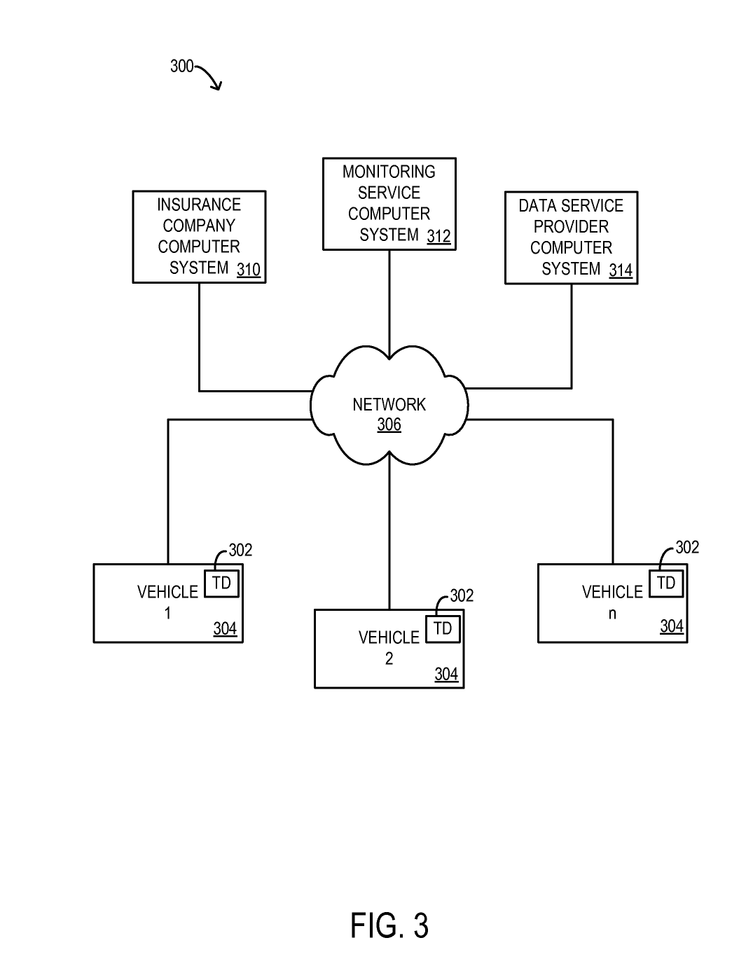

According to some embodiments, any or all of such data may be stored by or provided via one or more optional third-party data devices 106 of the system 100. A third-party data device 106 may comprise, for example, an external hard drive or flash drive connected to a server computer 102, a remote third-party computer system for storing and serving data for use in performing an underwriting function, or a combination of such remote and local data devices. In another example, the third-party data device 106 may comprise one or more telematics devices (such as the telematics devices 302 of FIG. 3 herein) associated with customer vehicles, as described hereinafter.

A third-party entity (e.g., a party other than an owner and/or operator, etc., of the server computer 102, customer computer 104, and other than an end-user of any data used in the underwriting process) may comprise, without limitation, (i) a third-party vendor, such as a monitoring service, which collects data from a telematics device (such as a telematics device 302 of FIG. 3 herein) associated with a customer's vehicle, or a data service provider, which aggregates vehicle data from various sources, a government agency, and/or a regulatory body, (ii) an insurance customer, and/or (iii) a demographic data gathering and/or processing firm.

A third-party entity, such as a monitoring service or a data service provider may, for example, collect and/or monitor vehicle data for various purposes deemed useful by the third party, including, without limitation, data mining, data analysis, data aggregation, price tracking, and/or sale or exchange of collected data. In one embodiment, any raw data, data analysis, and/or metrics may be stored on and/or made available (e.g., to an insurer) via the third-party data device 106. In one embodiment, one or more companies and/or end users may subscribe to or otherwise purchase data (e.g., vehicle data) from a third party and receive the data via the third-party data device 106. Such data may include vehicle data that has been measured or otherwise monitored, as well as data associated with vehicle identification numbers, claim history data, and/or data from state or federal government sources, such as motor vehicle departments, for example. Accordingly, in some embodiments, data from third-party sources, such as government and/or commercial entities, could be stored on, received from, and/or made accessible via third-party device(s) 106.

In some embodiments, the server computer 102 may store underwriting rules for writing and/or pricing insurance policies, and/or the customer computer 104 may execute the application remotely via the network 160 and/or download from the server computer 102 (e.g., a web server) some or all of the program code for executing one or more of the various embodiments described herein.

In one embodiment, a server computer 102 may not be necessary or desirable. For example, some embodiments may be practiced on one or more devices without a central authority. In such an embodiment, any functions described herein as performed by a server computer 102 and/or data described as stored on a server computer 102 may instead be performed by or stored on one or more such devices. Additional ways of distributing information and program instructions among one or more customer computers 104 and/or server computers 102 will be readily understood by one skilled in the art upon contemplation of the present disclosure.

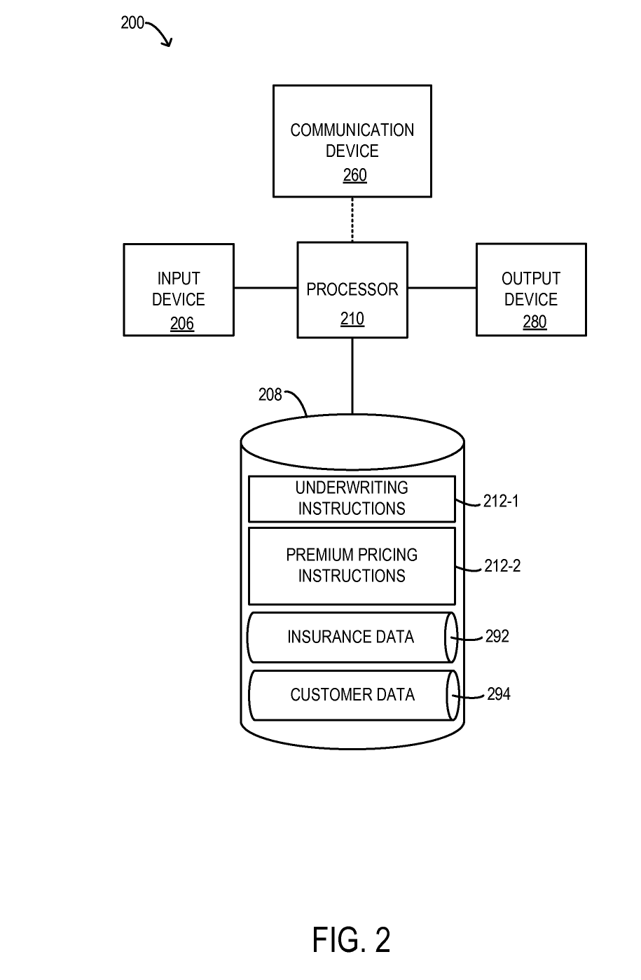

FIG. 2 depicts a block diagram of an apparatus 200 according to one embodiment. In some embodiments, the apparatus 200 may be similar in configuration and/or functionality to any of the customer computers 104, server computers 102, and/or third-party data devices 106 of FIG. 1. The apparatus 200 may, for example, execute, process, facilitate, and/or otherwise be associated with any of the processes described herein. In some embodiments, the apparatus 200 may comprise an input device 206, a memory device 208, a processor 210, a communication device 260, and/or an output device 280. Fewer or more components and/or various configurations of the components 206, 208, 210, 260, 280 may be included in the apparatus 200 without deviating from the scope of embodiments described herein.

According to some embodiments, the processor 210 may be or include any type, quantity, and/or configuration of processor that is or becomes known. The processor 210 may comprise, for example, an Intel® IXP 2800 network processor or an Intel® XEON™ Processor coupled with an Intel® E7501 chipset. In some embodiments, the processor 210 may comprise multiple inter-connected processors, microprocessors, and/or micro-engines. According to some embodiments, the processor 210 (and/or the apparatus 200 and/or other components thereof) may be supplied power via a power supply (not shown) such as a battery, an Alternating Current (AC) source, a Direct Current (DC) source, an AC/DC adapter, solar cells, and/or an inertial generator. In the case that the apparatus 200 comprises a server such as a blade server, necessary power may be supplied via a standard AC outlet, power strip, surge protector, and/or Uninterruptible Power Supply (UPS) device.

In some embodiments, the input device 206 and/or the output device 280 are communicatively coupled to the processor 210 (e.g., via wired and/or wireless connections and/or pathways) and they may generally comprise any types or configurations of input and output components and/or devices that are or become known, respectively.

The input device 206 may comprise, for example, a keyboard that allows an operator of the apparatus 200 to interface with the apparatus 200, for example, by an insurance customer or an insurance agent, such as to select parameters for monitoring by a telematics device (such as a telematics device 302 of FIG. 3 herein). In some embodiments, the input device 206 may comprise a sensor configured to provide information such as encoded measurement parameter data, vehicle data, or other customer information to the apparatus 200 and/or the processor 210.

The output device 280 may, according to some embodiments, comprise a display screen and/or other practicable output component and/or device. The output device 280 may, for example, provide information relating to premium discounts and/or surcharges, as well as information relating to premiums associated with one or more parameters and/or preferences a customer has selected for monitoring. According to some embodiments, the input device 206 and/or the output device 280 may comprise and/or be embodied in a single device such as a touch-screen monitor.

In some embodiments, the communication device 260 may comprise any type or configuration of communication device that is or becomes known or practicable. The communication device 260 may, for example, comprise a network interface card (NIC), a telephonic device, a cellular network device, a router, a hub, a modem, and/or a communications port or cable. In some embodiments, the communication device 260 may be coupled to provide data to a telecommunications device. The communication device 260 may, for example, comprise a cellular telephone network transmission device that sends signals (e.g., customer information, vehicle information, and/or a customer's selection of monitored vehicle parameters and/or monitoring preferences) to a server in communication with a plurality of handheld, mobile, smart phone, and/or other telephone devices. According to some embodiments, the communication device 260 may also or alternatively be coupled to the processor 210. In some embodiments, the communication device 260 may comprise an IR, RF, Bluetooth™, Near-Field-Communication (NFC), and/or Wi-Fi® network device coupled to facilitate communications between the processor 210 and another device (such as one or more customer computers, server computers, central controllers, and/or third-party data devices).

The memory device 208 (e.g., a computer-readable medium and/or computer-readable memory device) may comprise any appropriate information storage device that is or becomes known or available, including, but not limited to, units and/or combinations of magnetic storage devices (e.g., a hard disk drive), optical storage devices, and/or semiconductor memory devices such as Random Access Memory (RAM) devices, Read Only Memory (ROM) devices, Single Data Rate Random Access Memory (SDR-RAM), Double Data Rate Random Access Memory (DDR-RAM), and/or Programmable Read Only Memory (PROM).

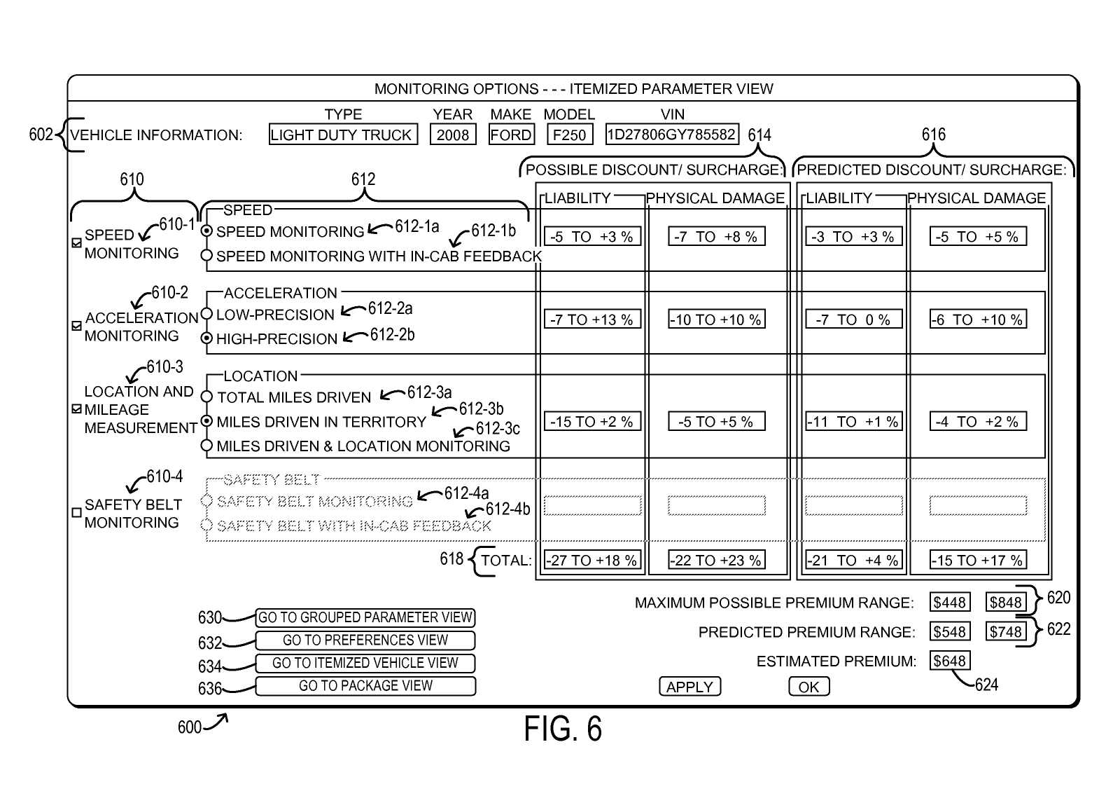

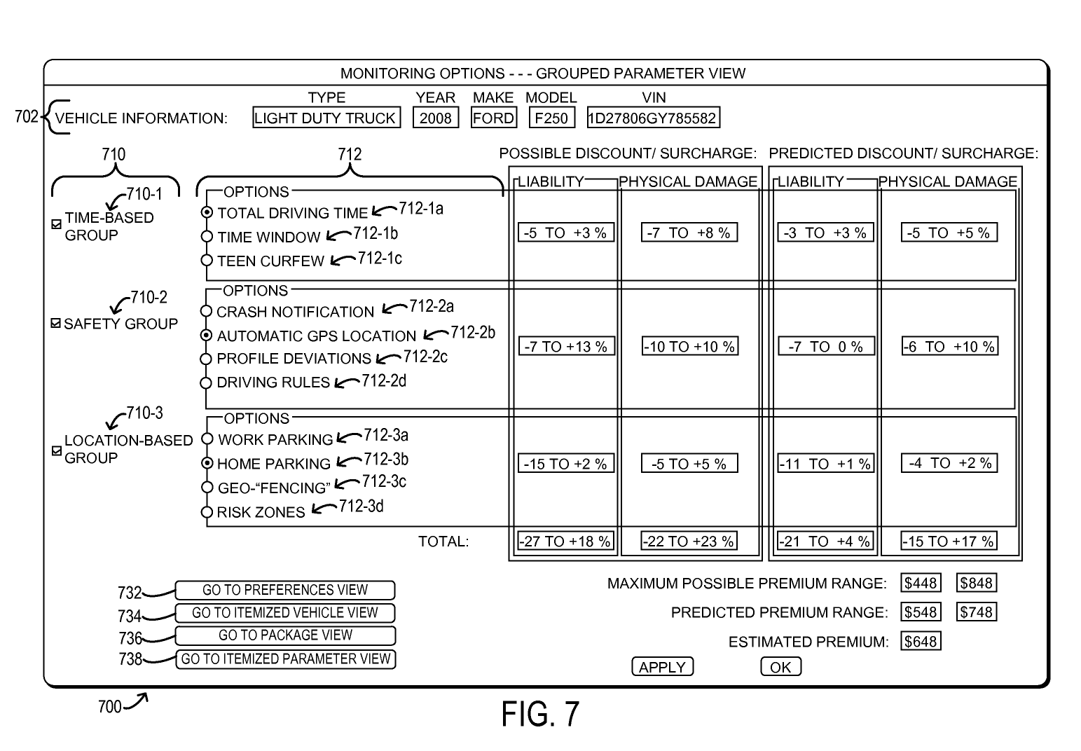

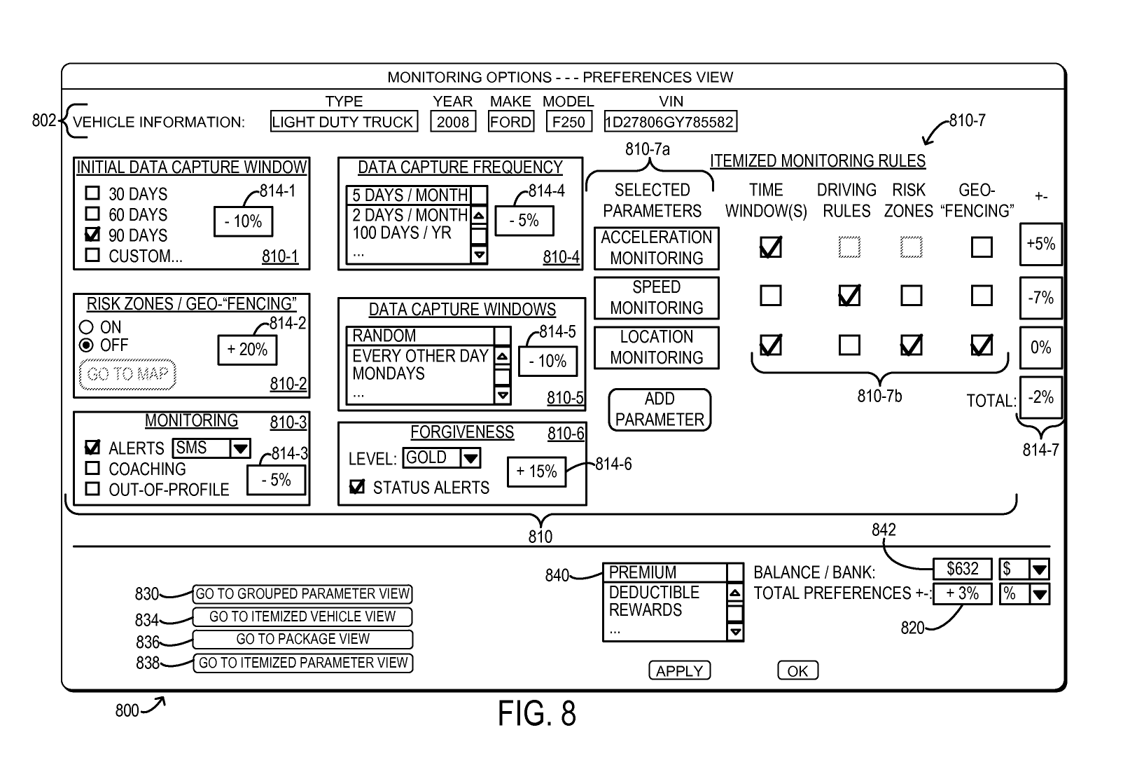

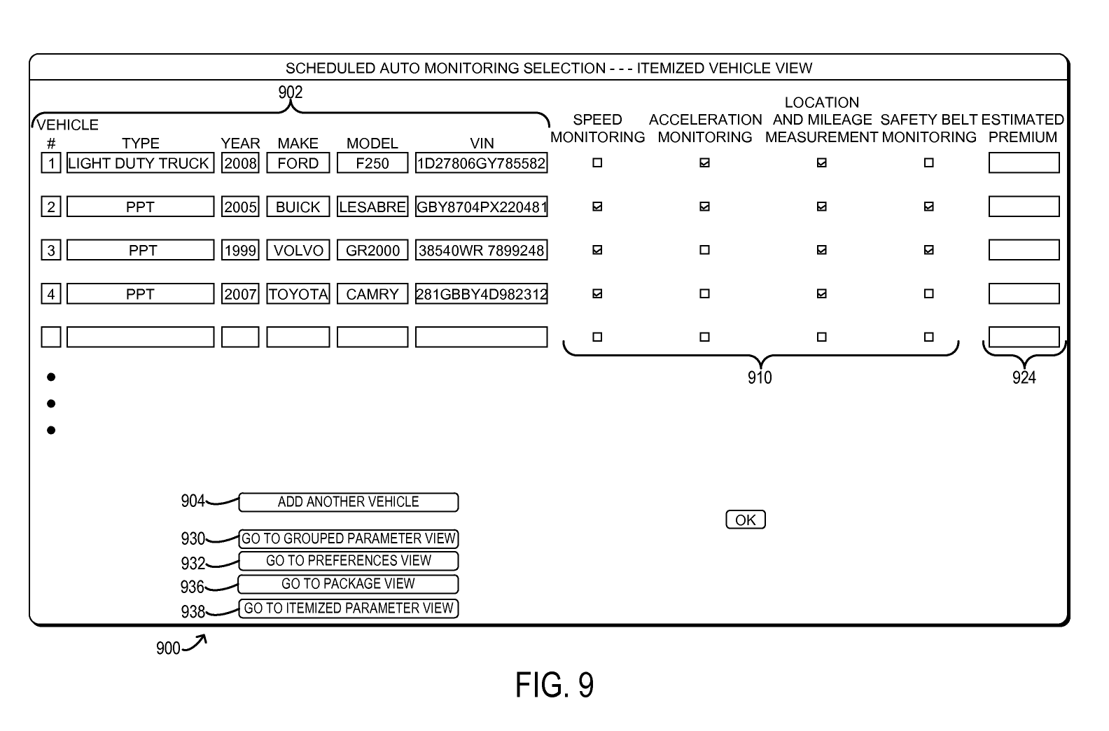

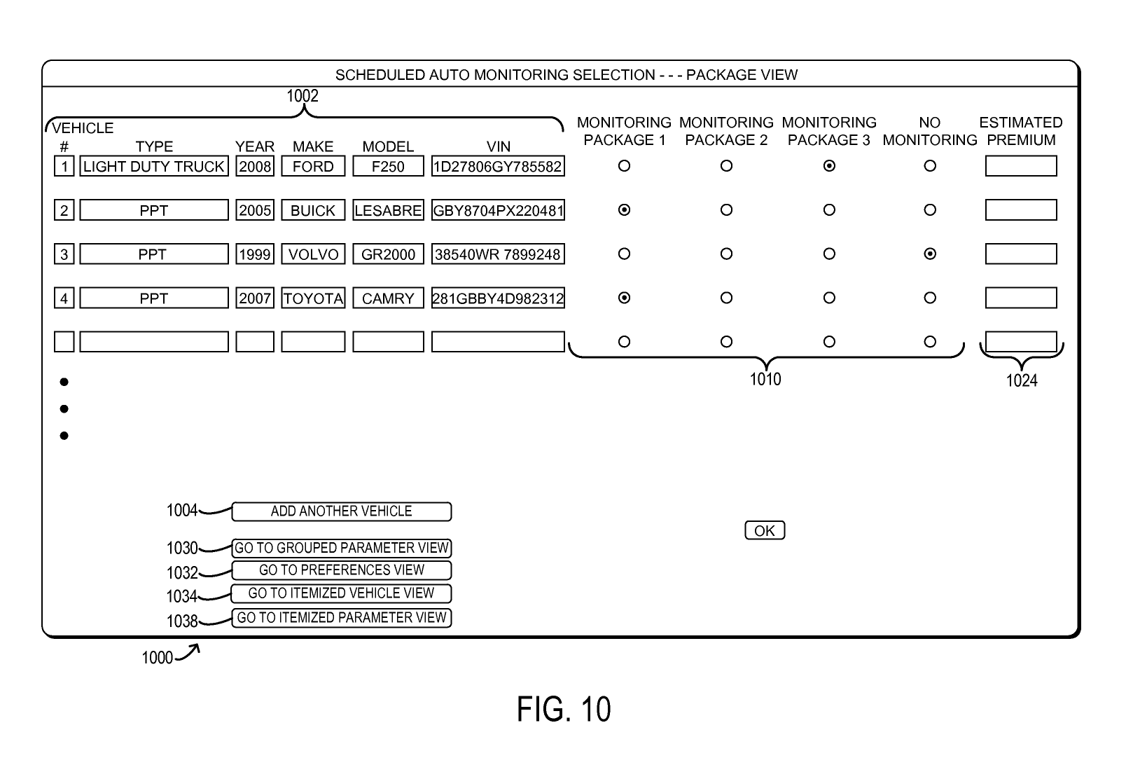

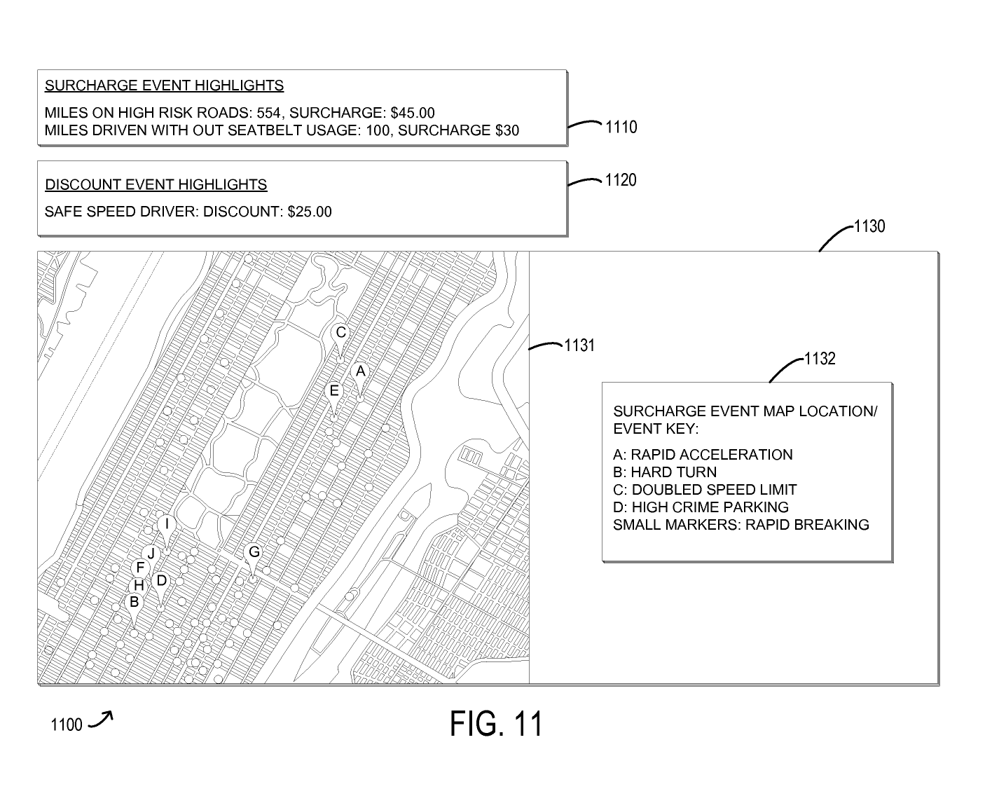

The memory device 208 may, according to some embodiments, store one or more of underwriting instructions 212-1, premium pricing instructions 212-2, insurance data 292, and/or customer data 294. In some embodiments, the underwriting instructions 212-1 and/or the premium pricing instructions 212-2 may be utilized by the processor 210 to provide output information via the output device 280 and/or the communication device 260 (e.g., via associated user interfaces, such as the example user interfaces 600, 700, 800, 900, 1000, 1100 of FIG. 6, FIG. 7, FIG. 8, FIG. 9, FIG. 10, and/or FIG. 11 herein).

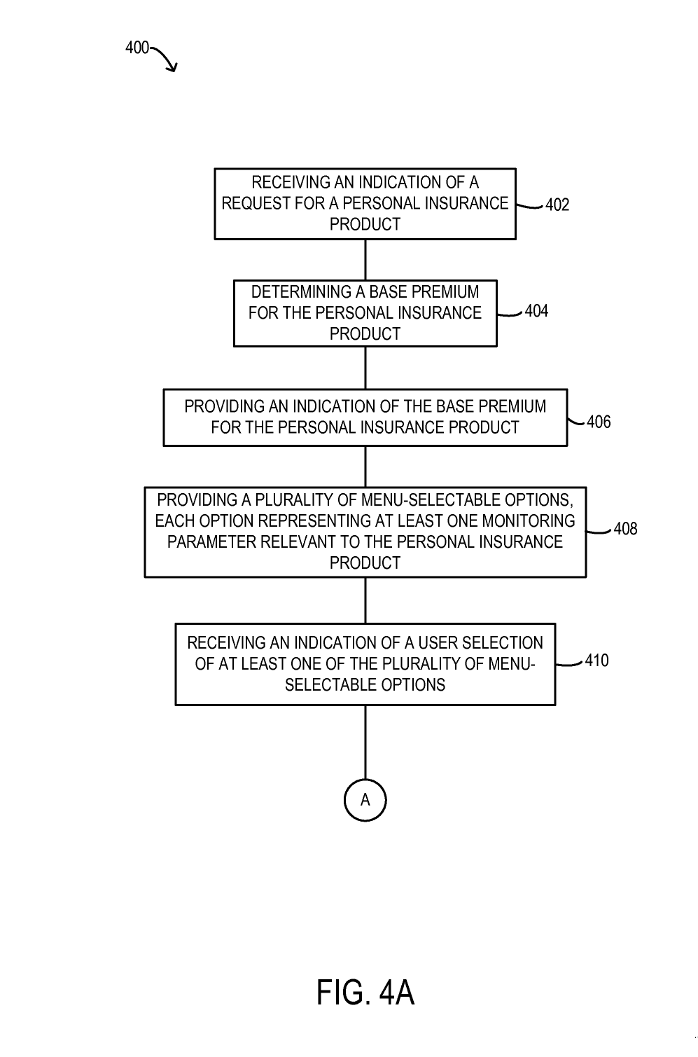

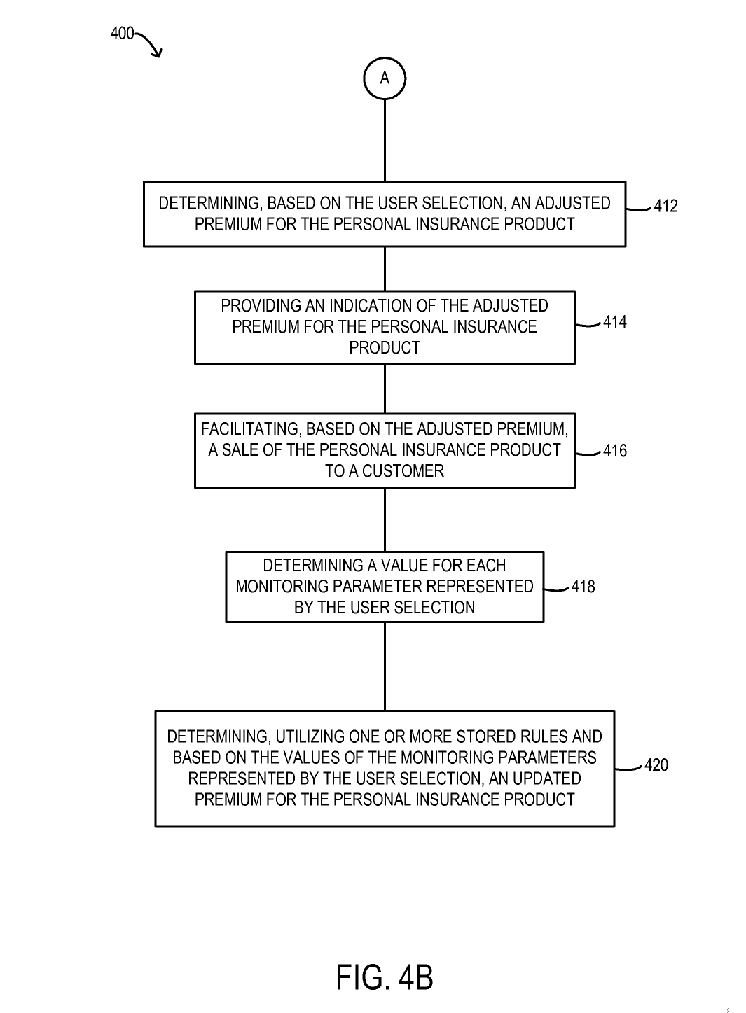

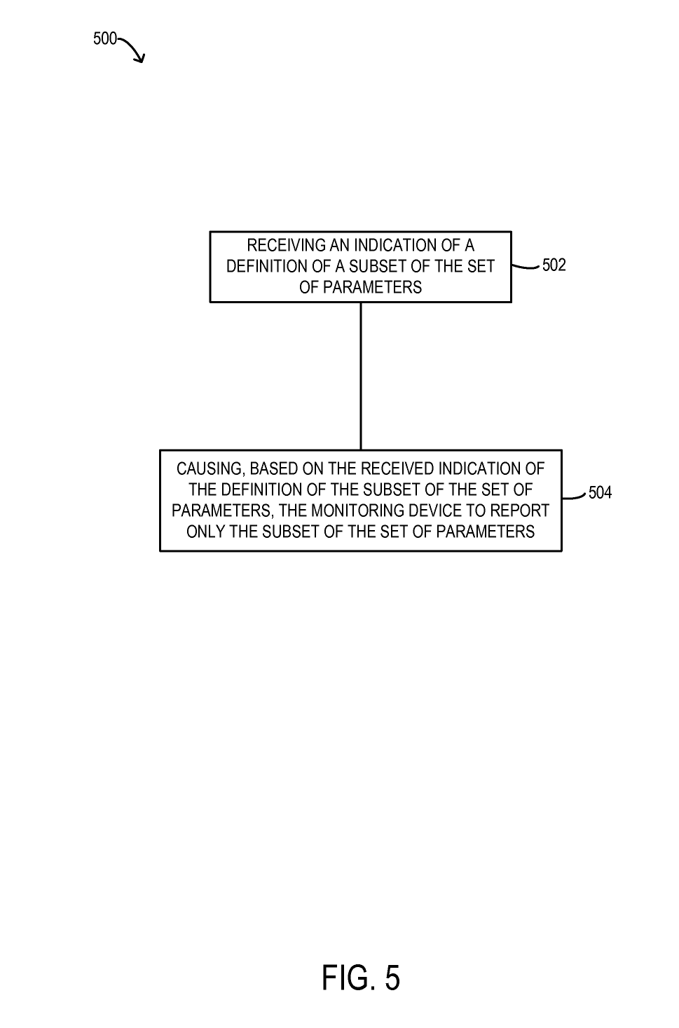

According to some embodiments, the underwriting instructions 212-1 may be operable to cause the processor 210 to process the insurance data 292 and/or the customer data 294 in accordance with embodiments described herein. Insurance data 292 and/or customer data 294 received via the input device 206 and/or the communication device 260 may, for example, be data mined, analyzed, sorted, filtered, decoded, decompressed, ranked, scored, plotted, and/or otherwise processed by the processor 210 in accordance with the underwriting instructions 212-1 (e.g., in accordance with the methods 400, 500 of FIG. 4A, FIG. 4B, and/or FIG. 5 herein). In some embodiments, insurance data 292 and customer data 294 may be fed by the processor 210 through one or more mathematical and/or statistical equations and/or models in accordance with the underwriting instructions 212-1 and/or premium pricing instructions 212-2 to define one or more discounts and/or surcharges that may then be utilized to provide premiums associated with one or more parameters that a customer has selected for monitoring.

According to some embodiments, the premium pricing instructions 212-2 may be operable to cause the processor 210 to perform a risk assessment (e.g., for an automobile insurance policy) in accordance with embodiments described herein. Insurance data 292 and/or customer data 294 may be analyzed to generate discounts and/or surcharges associated with parameters selected by the customer for monitoring and/or preferences for such monitoring, such as by a telematics device (e.g., the telematics devices 302 of FIG. 3 herein), for example. The underwriting instructions 212-1 and/or the premium pricing instructions 212-2 may, in some embodiments, utilize the insurance data 292 and/or the customer data 294 to provide an indication that an insurance policy should not be written or that a policy should be given a discount and/or a surcharge.

The apparatus 200 may function as a computer terminal and/or server of an insurance company that is accessible by an insurance agent and/or a customer, for example, and is utilized to determine discounts, surcharges, and/or premiums associated with one or more parameters that a customer has selected for monitoring and/or in accordance with preferences the customer has selected in relation to and/or defining how such monitoring should or shall occur. In some embodiments, the apparatus 200 may comprise a web server and/or other portal (e.g., an interactive voice response unit (IVRU)) that provides information on insurance policy discounts, surcharges, and/or premiums to users, agents, and/or customers.

Any or all of the exemplary instructions and data types described herein and other practicable types of data may be stored in any number, type, and/or configuration of memory devices that is or becomes known. The memory device 208 may, for example, comprise one or more data tables or files, databases, table spaces, registers, and/or other storage structures. In some embodiments, multiple databases and/or storage structures (and/or multiple memory devices 208) may be utilized to store information associated with the apparatus 200. According to some embodiments, the memory device 208 may be incorporated into and/or otherwise coupled to the apparatus 200 (e.g., as shown) or may simply be accessible to the apparatus 200 (e.g., externally located and/or situated).

FIG. 3 illustrates an exemplary telematics data system 300, in which telematics data is transmitted from a telematics device 302 in each of one or more vehicles 304 through a network 306 to an insurance company computer system 310 and, optionally, to one or more of a monitoring service computer system 312, and a data service provider computer system 314. The computer systems 310, 312, 314 may have an arrangement similar to the exemplary system 100 of FIG. 1 and/or may contain one or more apparatuses similar to the exemplary apparatus 200 of FIG. 2 herein. Other arrangements may also or alternatively be utilized. Further, the exemplary telematics data system 300 depicts three vehicles 304, but it is understood that such systems 300 according to embodiments may include fewer or more vehicles 304, including vehicles 304 of various types, as is or becomes desirable and/or practicable.

In one embodiment, telematics data is transmitted directly to the insurance company computer system 310, where the data is processed and analyzed (e.g., for insurance underwriting, modeling, quotation, quality review, risk analysis, loss analysis, and/or other purposes). In another embodiment, the data is transmitted first to a monitoring service computer system 312 where it is processed. The processed data is then transmitted to the insurance company computer system 310 where it is analyzed. In yet another embodiment, the telematics data is transmitted from the monitoring service computer system 312 to the data service provider computer system 314, where it is combined with other data, e.g., including third-party data, before being transmitted to the insurance company computer system 310. In other embodiments, the telematics data may be transmitted to one or more of the computer systems 310, 312, 314 simultaneously.

According to embodiments, a customer (not explicitly shown in FIG. 3) may select the data items that are monitored by the telematics device 302, and ultimately by the insurance company, and/or how such data items are to be monitored. Selection of data items and/or monitoring preferences by the customer may allow the customer to control the extent and/or nature of his or her discount, while overcoming any privacy restrictions that limit monitoring of personal information by third-parties. In one embodiment, the telematics device 302 may be configured to transmit only those data items selected by the customer and/or to only transmit such data items in accordance with any preferences and/or rules selected and/or otherwise defined by the customer. In another embodiment, the telematics device 302 may transmit a set of data items to a third party, such as a monitoring service or a data service provider, for example, but only the customer-selected data items may be provided by the third party to the insurance company, and/or they may only be provided in accordance with customer preferences (e.g., they may be filtered based on customer-defined or selected rules). In yet another embodiment, the telematics device 302 may transmit a full set of customer data to the insurance company, directly or via a third-party, and the insurance company may utilize only the customer-selected data items and/or may utilize the customer-selected preferences for determination of the customer's premium, discounts, surcharges, rewards, and/or penalties. Other arrangements may also be used as is or becomes desirable and/or practicable.

In some embodiments, the telematics device 302 may measure the data items directly from the vehicle 304. In one example, the telematics device 302 may communicate with the vehicle's onboard diagnostic (OBD) computer (not explicitly shown in FIG. 3), such as through an OBD port (e.g., OBD-II port) or comparable electrical connection. Such systems may allow the direct measurement of many aspects of the vehicle 304 and operation thereof. Other connections allowing direct vehicle measurements may also or alternatively be utilized. In such embodiments, the data may be actively transmitted by the telematics device 302 or it may be read from the telematics device 302, such as by an RFID and/or NFC scanner or other scanner (which are not depicted in FIG. 3), for example, as the vehicle 304 passes by and/or comes within proximity of the scanner.

In other embodiments, the telematics device 302 may provide information regarding the vehicle 304 and/or its operation by being present in the vehicle 304 and transmitting a signal while the vehicle 304 is operated without a connection to any vehicle system. In one example, such a telematics device 302 may comprise one or more of a Global Navigation Satellite System (GNSS) device, such as a Global Positioning System (GPS) device, a mobile phone or personal portable electronic device, an accelerometer, an RFID device, a trailer tracking device, and/or an intelligent vehicle device. Such devices 302 may be removably or fixedly mounted in the vehicle 304. In some embodiments, the monitoring service may be provided, for example, by the customer's mobile service provider (e.g., cell phone provider, 3G data service provider, etc.).

Still other embodiments may include telematics devices 302 that both measure vehicle parameters directly, as well as provide data by being present in the vehicle 304.

In some embodiments, certain data items may be determined indirectly, i.e., through other data items. For example, the amount of time a vehicle 304 spends in traffic may be determined using a combination of third-party data (e.g., posted speed limit) and measured speed and/or analysis of speed data (e.g., slow, frequent start/stop, etc.).

The telematics device 302 may include a remotely programmable memory (e.g., for designating the data items to monitor, record, and/or transmit, and/or for defining rules and/or preferences that govern how and/or when such data should be monitored, recorded, and/or transmitted), as well as various interfaces for wireless and/or hard-wired communications (e.g., for monitoring various aspects of driving and/or for communicating the monitored data items).

Telematics device vendors may comply with the data standard set by the insurance company or by industry standards organizations, such as the Association for Cooperative Operations Research and Development (ACORD), for example. In some embodiments, once the vendor indicates which standards of data transfer it will support, it may offer those options to the customer, who in turn may decide which transfer mode to use with the insurance company.

In some embodiments, all data from a given telematics device 302 may be monitored. For example, if accelerometer data is to be included, then that data may be used for any purpose, such as for detecting braking, swerving, acceleration, etc. In other embodiments, however, the customer may specify the types of behaviors to monitor. For example, although an accelerometer may be used to monitor both braking and swerving, a customer may choose to have one monitored and not the other. In another example, although GPS data may be used for multiple monitoring purposes, such as location, speed, time of day, etc., the customer may specify one or more specific data items to be monitored by GPS. In some embodiments, data to be monitored by the telematics device 302 may be governed by various rules and/or preferences. The accelerometer and/or GPS data may be recorded and/or tracked during certain time windows, with certain frequencies of measurement, with certain precision of measurement, and/or with respect to certain geographic parameters, that may, for example, be selected and/or defined by a customer (and/or third-party or insurer).

In addition, for each customer-selected data item, more than one method/device may be used to monitor the selected data item. For example, if the customer approved monitoring of speed, such monitoring may be achieved through different sources, such as the OBD connector to the vehicle computer and/or GPS data. In some embodiments, both may be considered approved by the customer under the speed-monitoring authorization.

As discussed above, embodiments may allow a customer to select the parameters (e.g., data items) monitored by an insurance company on which the customer's premium is based, instead of merely allowing the customer to opt in or opt out of a monitoring program. Also as described herein, embodiments may allow the customer to select how and/or when monitored parameters and/or data items (selected for monitoring by the customer, or otherwise) may be monitored, recorded, and/or transmitted.

An embodiment of a method 400 according to some embodiments is described with reference to FIG. 4A and FIG. 4B. As shown, the method 400 may comprise, at 402, receiving (e.g., by a specially-programmed computer device and/or from a user device) an indication of a request for an insurance product (such as a personal insurance product). The method 400 of this embodiment may further comprise, at 404, determining (e.g., by the specially-programmed computer device) a base premium for the requested insurance product. The base premium for the requested insurance product (and/or an indication thereof) may be provided in some embodiments, at 406, such as to a customer, for example.

In one embodiment, the insurance product may comprise a personal insurance product. As used herein, “personal insurance” generally relates to insurance policies owned by one or more individuals (e.g., families) on their own behalf, and not, for example, in association or affiliation with a business or other organization.

According to some embodiments, the method 400 may comprise providing a plurality of menu-selectable options, each option representing at least one monitoring parameter relevant to the requested insurance product, at 408. The menu-selectable options may be provided, for example, to a customer who has purchased or may purchase an insurance product (e.g., during an underwriting and/or quotation process). According to some embodiments, the monitoring parameters may comprise various driver, environmental, and/or vehicle data items such as, but not limited to, heart rate, respiration rate, head position (e.g., for determining “asleep-at-the-wheel” conditions), temperature, weather conditions, road conditions, traffic conditions, speed, acceleration, location, orientation, weight, fuel quantity (gas, diesel, Compressed Natural Gas (CNG), fuel cell, and/or battery charge/electricity) and/or engine or motor parameters. According to some embodiments, the monitoring parameters may also or alternatively comprise various preferences and/or rules regarding how and/or when various data items should be monitored, stored, recorded, transmitted, etc. For example, a customer may define and/or select one or more time windows, monitoring frequencies, geographic locations, barriers, and/or regions, and/or other parameters that govern and/or trigger data item monitoring, storing, recording, transmitting, and/or analysis. In some embodiments, the method 400 may comprise receiving an indication of a user selection of at least one of the plurality of menu-selectable options, at 410. The indication at 410 may be received, for example, via one of the example interfaces 600, 700, 800, 900, 1000, 1100 of FIG. 6, FIG. 7, FIG. 8, FIG. 9, FIG. 10, and/or FIG. 11 herein and/or via one or more of the components thereof (e.g., parameter-specific interface features).

In some embodiments, the method 400 may proceed, as shown at “A” in FIG. 4A, to “A” in FIG. 4B. In one embodiment, the method 400 may comprise determining, based on the user selection, an adjusted premium for the requested insurance product, at 412. The method 400 may, according to some embodiments, comprise providing an indication of the adjusted premium for the requested insurance product, at 414.

In one embodiment, the adjusted premium may comprise at least one of a presently discounted premium and an estimated future discounted premium. For example, some insurance programs provide incentives, such as discounts, for merely participating. Thus, once a user (e.g., a customer) selects one or more menu-selectable items for monitoring, that user may obtain a discount. In other insurance programs, discounts are not provided until a customer's driving characteristics have been monitored and found to be lower risk characteristics. In those cases, the adjusted premium may comprise an indication of a possible or predicted discount (e.g., a range of potential discount or other incentive levels).

In an embodiment, the method 400 may comprise facilitating, based on the adjusted premium, a sale of the requested insurance product to a customer, at 416. As used herein, “sale” may generally relate to the purchase of a policy by a new customer. It may also relate to the conversion or renewal of a policy by an existing customer. The method 400 may comprise, in some embodiments, determining a value for each monitoring parameter represented by the user selection, at 418. Insurance premium discounts and/or surcharges, and/or other rewards and/or penalties may be based on (e.g., looked-up and/or calculated utilizing) information regarding which parameters the customer desires to have monitored and/or based on what preferences the customer establishes regarding how parameters should be monitored (and/or otherwise utilized).

In one embodiment, the method 400 may comprise, at 420, determining, utilizing one or more stored rules and based on the values of the monitoring parameters represented by the user selection, an updated premium for the requested insurance product. According to an embodiment, the updated premium may comprise one of a discounted premium and a surcharged premium. For example, if a customer's driving characteristics are monitored and are found to be low risk, that customer may be given a discounted premium. On the other hand, if the customer is found to exhibit high-risk driving characteristics, the customer is likely to receive a surcharged premium.

According to some embodiments, instead of merely opting in or opting out of a monitoring program, a customer may select which monitoring parameters (i.e., data items) his insurance premium will be based on and/or how and/or when such parameters should be utilized. In one example, a customer may enroll in a vehicle insurance program and select the monitoring parameters to be monitored and/or for what timeframes, frequencies, and/or for what geographic locations such parameters should be monitored. An insurance premium may be calculated at least in part on the monitoring parameters and/or associated preferences that were selected (and/or defined) by the customer. For example, the insurance premium may be based on monitored values associated with the monitoring parameters and/or merely the customer's selection of certain data items and/or monitoring preferences thereof.

As described with reference to FIG. 3, the monitoring of a vehicle, according to embodiments, may be performed in various ways by various entities. For example, a telematics device 302 may be installed in a customer's vehicle 304 and may be monitored remotely by an insurance company computer system 310. In some embodiments, the telematics device 302 may be monitored by a monitoring service computer system 312, which relays the monitored information to the insurance company computer system 310. In some embodiments, a data service provider may, for example, aggregate data from various sources and send the aggregated data to the insurance company computer system 310. For example, the data service provider may receive the monitored data items from monitoring service computer system 312 as well as other information from, for example, a state division of motor vehicles, credit agencies, other monitoring services, customer reports, the federal government, etc. In some embodiments, the data service provider computer system 314 may filter out certain data items received from the monitoring service computer system 312. This filtering may be controlled, e.g., based on one or more of the identities of various parties (e.g., the customer, the monitoring service, the data service provider, and/or the insurance company) or based on any other options or data items that the parties select.

Turning to FIG. 5, a flowchart of a method 500 according to some embodiments is shown. In some embodiments, the method 500 may comprise receiving an indication of a definition of a subset of the set of parameters, at 502. The method 500 may also comprise causing, based on the received indication of the definition of the subset of the set of parameters, the monitoring device to report only the subset of the set of parameters, at 504.

The monitoring parameters presented to a customer for selection may, in some embodiments, depend on the type of telematics device 302 the customer is utilizing. For example, if a customer utilizes only a GPS device, the customer may be able to monitor either or both of speed and acceleration, but may not able to select airbag deployment monitoring. The options presented to a customer for selection may depend on information relating to the customer and/or the vehicle. For example, the available data items may depend on records retrieved from a state division of motor vehicles database or a database of the insurance company based on the vehicle's Vehicle Identification Number (VIN) or the customer's identification (e.g., driver license number and/or account identifier). If, for example, the vehicle records indicate the vehicle includes a Tire Pressure Monitoring System (TPMS) that may be monitored, the customer may be presented with the option to include tire pressure among the monitored data items. In another example, if the customer has many speeding violations, the insurance company may require the customer to allow his speed to be monitored as a condition for being insured through a telematics-based policy, or to receive a discount or qualify for a discount program for the policy.

According to some embodiments, the selection of monitoring parameters may be separated in steps performed by various customers or users of a vehicle. In the personal insurance context, such as in a family, for example, a husband may choose to have certain data items monitored when he uses a certain vehicle, while other family members may choose to have different data items monitored when they use the vehicle. In one embodiment, the telematics device 302 may include a user input device, such as a keypad, for example, with which the driver may identify who (e.g., family member) is driving. The device 302 may also or alternatively include a monitoring device, such as a biometric system or an RFID tag in a key or other device, to determine who is driving the vehicle. This capability may apply, e.g., to those drivers covered under the policy and those who are not and may be borrowing the vehicle. In addition, an individual driver may drive different vehicles. The parameters that are monitored may depend on which vehicle an individual is driving. Moreover, an individual may choose to have different data items monitored, depending on which vehicle he or she is driving. In some embodiments, rules associated with the parameters to be monitored may be defined, selected, and/or stored (e.g., by the customer). Vehicle speed may be monitored (e.g., as a required parameter or as selected by a customer), for example, only during certain time windows such as certain days of the week (e.g., workdays or weekends), certain times of the day (e.g., during work and/or commuting hours, during daylight hours, and/or during nighttime hours), and/or during certain seasons (e.g., winter driving). Similarly, parameters (such as speed) may only be monitored if certain conditions are met, such as, but not limited to, certain geographic conditions (e.g., a driver and/or vehicle is located in a particular area, zone, road, structure, or garage), certain environmental conditions (e.g., road conditions and/or weather conditions), and/or certain other conditions (such as legal conditions (e.g., teen driving laws), regulatory conditions (e.g., roadway vehicle type restrictions), and/or analytical conditions (e.g., deviations from a driver and/or vehicle profile)).

The monitoring parameters and/or monitoring rules or preferences may be combined in various ways for use in determining the insurance premium and/or other insurance services (e.g., risk control services). For example, a percentage discount or surcharge may be associated with each parameter and/or preference, and the percentages of each selected parameter/preference may be combined together to obtain a total percentage discount that will be deducted from or added to what would otherwise be the customer's premium, i.e., the “base premium.” In addition, various parameters, uses of the parameters, and/or preferences associated with the parameters may be grouped together into monitoring packages. Each of these packages may be assigned a percentage discount or surcharge. If multiple packages are selected, the associated discounts and/or surcharges may be combined together, but any duplicate discounts or surcharges for data items included in more than one selected package may be adjusted as appropriate.

In some embodiments, a premium may be affected by the mere selection of a parameter and/or the mere selection of a monitoring rule or preference. For example, the selection of each parameter and/or preference may result in a fixed, per data item discount or surcharge that is combined with others. In other embodiments, different parameters and/or preferences may result in different discounts or surcharges by, for example, assigning different weights to different parameters and/or preferences. In those embodiments, the weighting of the parameters and/or preferences may be based on how predictive of risk the parameters and/or preferences have been shown to be through analytical techniques. The customer may, in some embodiments, have the ability to designate the relative weighting of the data items. In other embodiments, a premium may depend on combinations of the selected parameters, selected preferences, and/or monitored values associated with those selected parameters and/or preferences. In such embodiments, different parameters and/or preferences may be given different weights, which then may vary further depending on the monitored values associated with those parameters/preferences.

The relationship between a premium and a monitored value may be based on various functions, e.g., linear, stepped, or a smoothly changing slope. Using speed in excess of a speed limit as an example, one brief period of time over a speed limit may have no effect on the premium, two (2) five-minute (5-min) periods may result in a one percent (1%) surcharge, and four (4) five-minute (5-min) periods may result in a three percent (3%) surcharge. In another embodiment, once ten (10) five-minute (5-min) periods in excess of the speed limit are detected, this parameter may have no further effect on the premium, regardless of how many more times a customer drives in excess of the speed limit. In other cases, each five-minute (5-min) period in excess of the speed limit may result in a constant surcharge that continually increases for every detected period. In addition, out of tolerance measurements for certain combinations of parameters may have an enhanced effect on discounts and/or surcharges. For example, a surcharge for speeding may be increased beyond its normal value if it is also detected that the seat belt is not in use and/or the vehicle is out of maintenance (e.g., the vehicle's tire pressure is low). In another example, speeding in certain areas (e.g., large rural highways with few intersections or exits) may result in a lesser surcharge than speeding in other areas (e.g., a crowded urban areas with many intersections).

Exemplary monitoring parameters and exemplary uses of those parameters in calculating an insurance premium or a discount and/or surcharge to a premium include, but are not limited to, those listed below. Other parameters may also be utilized. In addition, parameters indicated as providing a discount may result in a surcharge in certain circumstances.
Mileage: a discount applied to a premium determined by a defined relationship with the total miles traveled in a specific period of time, or the type of miles driven, such as fraction of miles driven on highways or rural roads.
Speeding: a discount applied to a premium determined by a defined relationship with the number of events in which the vehicle's speed exceeds a predetermined threshold, based on at least one of absolute speed of the vehicle, speed of the vehicle in relation to the posted speed limit on a traveled roadway, and the speed of the vehicle in relation to the speed of other vehicles on the traveled roadway in a specific period of time.
Observing traffic control: a discount applied to a premium if an emergency management call system is activated, or active use of a traffic congestion monitor for areas, such as urban road networks, freeway networks, and ability to change traffic routes using a traffic guidance/avoidance system.
Hard Braking: a discount applied to a premium determined by a defined relationship with the number of events where the vehicle's rate of deceleration (braking) exceeds a predetermined threshold in a specific period of time, or where the vehicle's rate of deceleration exceeds a variable threshold in a specific period of time based on the vehicle's speed at the start of deceleration.
Hard Cornering: a discount applied to a premium determined by a defined relationship with the number of events where the vehicle's speed during specific driving maneuvers exceeds a predetermined threshold in a specific period of time, where the lateral acceleration exceeds a predetermined threshold in a specific period of time, or where the lateral acceleration exceeds a predetermined variable threshold in a specific period of time based on the type of cornering and/or location, such as an expressway ramp or an intersection.
Hard Acceleration: a discount applied to a premium determined by a defined relationship with the number of events where the vehicle's rate of acceleration exceeds a predetermined threshold in a specific period of time, or where the vehicle's rate of acceleration exceeds a variable threshold in a specific period of time based on the vehicle's speed at the start of acceleration.
Swerving/Erratic: a discount applied to a premium determined by a defined relationship with the number of events where the lateral movement of a vehicle while traveling in a traffic control lane exceeds a predetermined threshold in a specific period of time, or where the lateral movement of a vehicle equipped with lane guidance systems exceeds a predetermined threshold in a specific period of time given lane guidance system readout information.
Seat Belt usage: a discount applied to a premium determined by a defined relationship with the number of events where the operator's and passenger's seat belts are not utilized during vehicle travel exceeds a predetermined threshold in a specific period of time.
Turn signal usage: a discount applied to a premium determined by a defined relationship with the number of events where a vehicle's turn signal is not used prior to the initiation of specific driving maneuvers requiring their use in a specific period of time.
Tailgating: a discount applied to a premium determined by a defined relationship with the number of events where the distance between the vehicle and a vehicle directly front of it is less than a specified threshold in a specific period of time, or where the distance between the vehicle and a vehicle directly in front of it is less than a variable threshold based on the following vehicle's speed in a specific period of time.
Green Driving: a discount applied to a premium determined by a defined relationship where the vehicle's carbon emissions are less than a defined threshold in a specific period of time, where the vehicle's speed and acceleration patterns given the road type indicate efficient driving, or where a green driving system (e.g., OEM, mobile application, etc.) indicates green driving behavior above a defined threshold over a specific period of time.
Distraction: a discount applied to a premium determined by a defined relationship between the number and duration of events where the operator is distracted in the course of operating a motor vehicle as indicated through in-cab video (e.g., “eyes up” technology) or other means (e.g., monitoring operator's use of a cellular device) is less than a specific threshold in a specific period of time, or where the operator utilizes one or more systems to minimize distraction while driving, such as cell phone signal blocking applications.
Near collision: a discount applied to a premium determined by a defined relationship which the number and duration of events where the a near-collision noted in a video system or other means, but not otherwise indicated in other telematics systems is less than a specific threshold in a specific period of time.
Naturalistic behavior: observance of behavior of the driver or other key individuals in their own setting that may be indicative of risk.
Total driving time: a discount applied to a premium determined by a defined relationship with the total seconds a vehicle is in motion in a specific period of time. Time a vehicle is in motion may be determined by measurements when the vehicle is in motion (e.g., using phone, GPS, etc.) and/or when the vehicle at rest (e.g., measuring the amount of time the engine is turned off).
Time of day: a discount applied to a premium determined by a defined relationship where the total seconds a vehicle is in motion during specific periods of time and/or during specific days of the week exceeds a predetermined threshold. This may be particularly applicable, for example, for hired vehicles (e.g., independent contractors, owner operator, etc.).
Location: a discount applied based on where a vehicle for the majority of the time, such as: Work parking: if your vehicle is located at your place of employment or parking garage associated with your employment; Home parking: if your vehicle is located at your place of residence (garage, carport, or open air); Location-driven: depending on where your vehicle is driven (e.g., rural roads, urban or highway), where the vehicle is driven most often (e.g., average zip code of vehicle location based on total seconds the vehicle is in motion), or a combination of prevalent locations where the vehicle is parked and/or is driven.
Weather: a discount applied based on time spent or performance while driving in certain weather conditions. In some embodiments, external weather conditions such as temperature, humidity, pressure, etc. may be measured. Alternatively and/or in addition, third party data (e.g., NOAA data) may be used to determine weather conditions.
Behavioral: an aspect of the driver's overall behavior that may be indicative of losses when considered alone or in combination with other measured behaviors.
Drug/Alcohol usage: a discount applied based on measurement of drug or alcohol level (e.g., breathalyzer).
Fatigue: a discount applied based on the driver not being fatigued while driving, as determined by fatigue sensors, or where the vehicle is driven less than a determined threshold during particular times of day or days of week where prevalence of fatigue is higher than a predetermined threshold.
Maintenance: a discount applied based on the maintenance level of the vehicle (e.g., engine light on, car status report based on parameters available from vehicle's OBD port, tire pressure monitor, engine temperature monitor, airbag monitor, seatbelt monitor, window position monitor, door monitor, visibility monitor, in-cab or external video feed, the vehicle's electrical, mechanical, and emissions systems, etc.)
Application usage: a discount applied to a premium determined by a defined relationship with the usage of specified software applications (e.g., smart phone apps, automatic car start, or other features/tools).

As described above, various data items and uses of the data items may be grouped into packages. Packages may be, for example, time-based, location-based, time- and location-based, safety-based, high-risk, green, teen driving, carpool/commuter, sleeping, DUI, distracted driver, and various combinations thereof. Some examples are provided below, that may be based on, for example, data items described above and additional data items described below.
Safety package: based on the type of safety devices (e.g., in the event of a collision in which the airbags are deployed, or whether airbags deploy and an automatic crash response is sent to assist emergency response efforts), or where an operator utilizes driver feedback or other systems to modify driving behavior to increase safe driving characteristics, whether real-time or through post-driving reports and feedback.
High risk package: based on a determination if high risk driving has or could occur. This may include time spent driving during particular times of day or days of week, time spent driving during predetermined weather conditions, time spent driving during particular traffic congestion conditions, or a combination of driving behavior factors that are indicative of risky driving behavior.
Green Package: based on a determination of the types of miles driven, CO₂ emissions, O₂ sensor readings, vehicle's mileage per gallon, greenhouse gas emissions, materials vehicle is constructed of, utilization of green driving systems, such as real-time feedback to driver to facilitate green driving behavior, or amount of time vehicle is idling.
Teen Driving Package: based on a determination of the types of educational courses completed, types of telematics devices installed, types of distraction-free technologies employed, types of miles driven, and time of day the vehicle is driven during the teen operations, or based on usage of vehicle monitoring systems such as geo-fencing or time-of-day alerts via text message, e-mail, or other immediate communication to owner of vehicle.
Carpool/Commuter Package: based on a determination if the vehicle operator, during normal business hours (day or evening), is considered to be a commuter or uses an official carpooling service.
Time Windows Package: monitoring and/or utilization of certain and/or various parameters limited to and/or excluded from (and/or otherwise based on) certain times, times of year, seasons, and/or days of the week (e.g., day only, night only, off-peak only, weekend only).
Risk Zones Package: monitoring and/or utilization of certain and/or various parameters limited to or excluded from (and/or otherwise based on) one or more road segments, intersections, traffic and/or parking structures or areas, districts, areas, and/or other location-based zones or data points.
Busyness Package: monitoring and/or utilization of certain and/or various parameters limited to or excluded from (and/or otherwise based on) one or more indicators of busyness (e.g., as described in co-pending U.S. patent application Ser. No. 12/978,535 filed on Dec. 24, 2010 and titled “RISK ASSESSMENT AND CONTROL, INSURANCE PREMIUM DETERMINATIONS, AND OTHER APPLICATIONS USING BUSYNESS”, the “busyness” concepts and descriptions of which are hereby incorporated by reference herein).
Sleeping Package: based on a determination if the vehicle operator, during hours the vehicle is in motion (day or evening), is sleeping, or drowsy.
DUI Package: based on determination if a vehicle operator is deemed safe to operate a vehicle without the impairment of alcohol or legal/illegal pharmaceuticals.
Distracted Driving Package: based on a determination of the types educational courses completed, types of telematics devices installed, types of distraction-free technologies employed, types of miles driven, types of in-vehicle driver feedback employed (e.g., cell phone usage, in-cab video, etc.), number of occupants in the vehicle, and/or time of day the vehicle is driven during the vehicle's operations.

According to some embodiments, based on the monitoring parameters and/or preferences selected, and/or the values measured for the respective monitoring parameters, the insurance company may underwrite a given customer into a respective product, program, and/or company. A product, sometimes referred to as a program or package (the terms are used interchangeably herein), may generally comprise a marketing concept that represents an offering to a customer. In various embodiments, the product (or program or package) may be a collection of features, which includes a particular pricing plan, particular benefits, limitations, qualifications, etc. The product (or program or package) may have its own unique insurance contract and/or may use the same insurance contract as other products. Customers who qualify may, for example, be placed into an “experienced driver product” or a “safe teens product,” etc. A product may be available through one or many companies, but it should generally be associated with at least one company to be a viable product, because the insurance contract is written under the auspices of that company. Underwriting into a company refers to the insurance company using data to determine which one, among several different subsidiary companies doing business in a state, to place the customer into. In some cases, based on the monitoring parameters and/or preferences, it may be necessary to underwrite into a different product within the same company. In other cases, it may be necessary to place the customer with a new company altogether.

In an exemplary interaction between a customer and an insurance company, the customer may learn about the insurance telematics program through advertisements (e.g., television, internet, radio, etc.), solicitation from an agent, and/or solicitation through mail, for example. The customer may utilize an on-line quoting and issuance system to determine possible quotes and input demographic information. The insurance system may configure and display potential customer discounts or surcharges based on, e.g., packages or various selected data items and/or preferences. The customer may, for example, select itemized parameters for monitoring (i.e., specific desired data items), preferences defining when and/or how such parameters should be monitored and/or utilized, one or more packages of parameters, a combination of itemized parameters and packages, or no monitoring at all. The customer may make these various selections based on, e.g., individual drivers, various vehicles, groups of drivers, driver-vehicle combinations, etc. The customer may then finalize the selections and the insurance company may quote and/or issue a policy.

One or more telematics devices (such as the telematics devices 302 of FIG. 3 herein) may be configured based on, for example, the customer selections. The customer may view the possible discounts and/or surcharges and selections, and adjust the selections during the insurance policy term. Such adjustments may be carried out, for example, by using a web portal, speaking or communicating with an agent, receiving an automatically generated report or bill, and/or receiving a vendor report. When a customer, for example, changes the monitored selections, the system may reconfigure its settings and reconfigure the telematics device as described herein. In some embodiments, the customer may affect his insurance premium discount or surcharge by, for example, changing driving behavior or opting out of the program.

According to some embodiments, a telemetric monitoring device may be operable to monitor a plurality of telemetric parameters. The device may comprise an electronic processing device, a wireless communication device in communication with the electronic processing device, and/or a remotely programmable memory in communication with the electronic processing device and the wireless communication device.

In some embodiments, the programmable memory stores instructions that when executed by the electronic processing device may result in execution of various procedures and/or methods. In FIG. 5, for example, a method 500 according to some embodiments may comprise determining (e.g., based on an instruction from a remote device) a subset of a plurality of telemetric parameters that are to be monitored, at 502. The instructions, when executed, may further result in monitoring the determined subset of the plurality of telemetric parameters, storing, by the remotely programmable memory, information descriptive of the monitored subset of the plurality of telemetric parameters, and/or transmitting, by the wireless communication device and to a remote server device, an indication of the information descriptive of the monitored subset of the plurality of telemetric parameters. In some embodiments, the method 500 may comprise causing, based on the received indication of the definition of the subset of the set of parameters, the monitoring device to report only the subset of the set of parameters, at 504. In such a manner, for example, a telematics device may be customized based on customer preferences. In some embodiments, customer selected and/or defined constraints, rules, and/or triggers may also or alternatively be set via remote programming of the telematics device. Various time-windows, geographic constraints, and/or environmental triggers may be set, for example, that cause the telematics device to adjust operations (e.g., on/off, monitor/don't monitor) based on the customer-defined preferences.

In some embodiments, the stored instructions, when executed by the electronic processing device, may result in receiving (e.g., via the wireless communication device) the instructions from a remote device. In a further embodiment, the remote device may comprise a smart phone operated by a customer. In yet another embodiment, the remote device may comprise a key fob storing information identifying a customer. In a still further embodiment, the remote device and the remote server device may be the same.

In some embodiments, the telemetric monitoring device may comprise a sensor device coupled to a vehicle and the subset of the plurality of telemetric parameters may comprise one or more of: (i) a vehicle speed parameter; (ii) a vehicle door status parameter; (iii) a vehicle window status parameter; (iv) a vehicle seatbelt status parameter; (v) a vehicle acceleration parameter; (vi) a vehicle braking parameter; (vii) a vehicle airbag status parameter; (viii) a vehicle tire pressure parameter; (ix) a vehicle usage parameter; (x) a vehicle turn signal status parameter; (xi) a vehicle engine parameter; (xii) a vehicle location parameter, (xiii) a monitoring time-window, (xiv) a monitoring geographic and/or location rule, and/or (xv) an environmental monitoring trigger.

In some embodiments, the telemetric monitoring device may comprise a sensor device coupled to sense a driver of a vehicle and the subset of the plurality of telemetric parameters may comprise one or more of: (i) a driver distraction parameter; (ii) a driver fatigue parameter; and (iii) a driver substance abuse parameter.

FIG. 6, FIG. 7, FIG. 8, FIG. 9, FIG. 10, and FIG. 11 depict exemplary user interfaces 600, 700, 800, 900, 1000, 1100 that may presented to and used by prospective customers, current customers, and/or insurance agents and/or insurance company employees, e.g., to develop premium estimates/quotes, select data items, update selected data items, select and/or define monitoring preferences, update selected and/or defined monitoring preferences, and/or update the identification of participating individuals, entities, and/or vehicles. The exemplary user interfaces may be presented on a browser, such as a web browser or a Wireless Application Protocol (WAP) browser on a customer's home computer or mobile device (e.g., phone, PDA) over a connection to a network (e.g., the network 306 of FIG. 3), for example, or on a computer that is part of an insurance company computer system (e.g., the insurance company computer system 310 of FIG. 3). Other arrangements may also or alternatively be utilized. In some embodiments, instead of or in addition to browser windows, customers may configure their policies (e.g., update their selected parameters and/or preferences) utilizing messaging, such as text messaging over SMS. In some embodiments, the user interfaces 600, 700, 800, 900, 1000, 1100 may be provided by an application running on a customer's mobile device and/or may be integrated into the customer's vehicle, such as being incorporated into the vehicle's trip computer and/or onboard navigational system (OEM and/or after-market).

In various embodiments, the updates discussed herein may result in real time updates to the system settings for monitoring parameters and/or preferences. Thus, customers may possess a high degree of control over the selection of data items that are monitored, recorded, transmitted, and/or utilized, and/or how and/or when such parameters are monitored, recorded, transmitted, and/or utilized, as a basis for insurance premium calculation. For example, if a customer updates a selection of parameters to be monitored for a specific vehicle (and/or preferences governing such monitoring), the system may program a telematics device monitoring that vehicle in real time. In some embodiments, the customer may receive confirmation not only of his selected data items and/or preferences, but also that a telematics device has been programmed in accordance with those selected data items. For example, if the customer sends an SMS text message indicating that his location should no longer be monitored (e.g., a geographic and/or location “stop monitoring” trigger—one-time or for recurrence), the system may program a telematics device associated with his vehicle to stop monitoring location and send the customer an SMS or other text message indicating that the telematics device has been reprogrammed per his instructions. In addition, different individuals who share a vehicle may login to a telematics device of that vehicle when, for example, they enter the vehicle. Moreover, the individual's login may cause the system to reprogram the telematics device (and/or, e.g., the monitoring service computer or insurance company computer's data item filtering system) to conform to that individual's settings in real time. In addition, the individual may be notified, e.g., by SMS message, that the reprogramming has been performed.

FIG. 6 illustrates an itemized parameter selection screen interface 600 that may be utilized, for example, by a customer through a web portal, smart-phone application, in-vehicle device, interactive voice response unit (IVRU), or by an insurance agent on the phone with a customer. As shown, the customer may select (e.g., for one or more specific vehicles 602 (and/or drivers or combinations thereof)) one or more parameters to be monitored 610 at the left of the screen (speed 610-1, acceleration 610-2, location and mileage 610-3, and/or safety belt 610-4). Within each of these example parameters 610, there are sub-items 612 shown to the right of the parameters (e.g., speed monitoring 612-1a, speed monitoring with in-cab feedback 612-1b, low-precision acceleration monitoring 612-2a, high-precision acceleration monitoring 612-2b, total miles driven 612-3a, miles driven in territory 612-3b, miles driven and location monitoring 612-3c, safety belt monitoring 612-4a, and/or safety belt monitoring with in-cab feedback 612-4b). For example, if speed monitoring 610-1 is selected, the customer may select in-cab feedback 612-1b if desired. In some embodiments, such as if, for example, the data item 610 is not selected, then the sub-items 612 may not be selected, and are displayed in a lighter shade of text (e.g., as shown by the safety belt monitoring sub-items 612-4). Other sets of parameters and sub-items may also be used.

In the illustrated embodiment, a first set of discount/surcharge columns 614 show the maximum possible percentage ranges of discount/surcharge that selection of data items 610 may result in (for each of liability and physical damage). For example, the indicated speed monitoring selection 610-1, 612-1a could result in between a negative five percent (−5%) and positive three percent (+3%) change in a liability component of a premium and a negative seven percent (−7%) to positive eight percent (+8%) change in the physical damage component of the premium. The second set of discount/surcharge columns 616 show the predicted discount range(s) based on, for example, drivers with similar profiles to the current customer. For example, the indicated speed monitoring selection 610-1, 612-1a may be predicted to result in between a negative three percent (−3%) and positive three percent (+3%) change in the liability component of the premium and a negative five percent (−5%) to positive five percent (+5%) change in the physical damage component of the premium. The discounts of each column 614, 616 are totaled in the “Total” row 618 at the bottom of the columns 614, 616.

In some embodiments, the maximum possible premium range 620, the predicted premium range 622, and the estimated premium in dollars 624 are shown along the bottom of the screen. In this example, the customer's maximum possible premium range 620 is between four hundred and forty-eight dollars ($448) and eight hundred and forty-eight dollars ($848); the predicted premium range 622 is between five hundred and forty-eight dollars ($548) and seven hundred and forty-eight dollars ($748); and the estimated premium 624 is six hundred and forty-eight dollars ($648).

If the customer would like to see the effect that allowing the monitoring of various parameters 610 or combinations of parameters 610 has on the insurance premium (e.g., the estimated premium 624), the customer may enter one or more parameters 610 and/or sub-items 612 and click on “APPLY” to populate the chart. Once the customer is satisfied with the selections, the settings may be entered by clicking “OK.” In some embodiments, buttons or other navigational aids or tools may be provided to allow the customer to switch views and/or go to other data screens, such as the “Go to Grouped Parameter View” button 630 (selection of which, e.g., may cause the interface 700 of FIG. 7 to be provided), the “Go to Preferences View” button 632 (selection of which, e.g., may cause the interface 800 of FIG. 8 to be provided), the “Go to Itemized Vehicle View” button 634 (selection of which, e.g., may cause the interface 900 of FIG. 9 to be provided), and/or the “Go to Package View” button 636 (selection of which, e.g., may cause the interface 1000 of FIG. 10 to be provided).

FIG. 7 illustrates an embodiment of a grouped parameter view selection screen interface 700 that may be utilized in a similar manner to the user interface 600 shown in FIG. 6. In the illustrated example, the customer has selected (e.g., for one or more specific vehicles 702 (and/or drivers or combinations thereof)) three (3) parameter groups 710 (e.g., and indications of such selection(s) have been received): a time-based group 710-1, a safety group 710-2, and a location-based group 710-3. In some embodiments, multiple option selections 712 may be made within each set of parameter groups 710 (e.g., total driving time option 712-1a, time window option 712-1b, teen curfew option 712-1c, crash notification option 712-2a, automatic GPS location option 712-2b, profile deviations option 712-2c, driving rules option 712-2d, work parking option 712-3a, home parking option 712-3b, geo-“fencing” option 712-3c, and/or risk zones option 712-3d). For example, it may be possible to select both the work parking option 712-3a and the home parking option 712-3b. Additional or alternative options 712 (not shown in FIG. 7) may include, e.g., constant location monitoring or parked location monitoring. Other arrangements of parameters 710 and/or options 712 may also or alternatively be utilized. In some embodiments, buttons or other navigational aids or tools may be provided to allow the customer to switch views and/or go to other data screens, such as the “Go to Preferences View” button 732 (selection of which, e.g., may cause the interface 800 of FIG. 8 to be provided), the “Go to Itemized Vehicle View” button 734 (selection of which, e.g., may cause the interface 900 of FIG. 9 to be provided), the “Go to Package View” button 736 (selection of which, e.g., may cause the interface 1000 of FIG. 10 to be provided), and/or the “Go to Itemized Parameter View” button 738 (selection of which, e.g., may cause the interface 600 of FIG. 6 to be provided).

Referring now to FIG. 8, an example preferences view interface 800 according to some embodiments is shown. In some embodiments, the interface 800 may be configured to permit a customer (and/or other user) to select and/or define (e.g., for one or more specific vehicles 802 (and/or drivers or combinations thereof)) various preferences 810 relating to telematic monitoring. As shown in the example interface 800, such preferences 810 may comprise (but are not limited to), initial data capture window preferences 810-1, risk zones/geo-“fencing” preferences 810-2 (and/or busyness preferences), monitoring preferences 810-3, data capture frequency preferences 810-4, data capture windows preferences 810-5, and/or forgiveness preferences 810-6. In some embodiments, the interface 800 may also or alternatively be configured to allow a customer to manage a matrix of preferences 810-7. For a list of selected parameters 810-7a, for example, one or more rules 810-7b (e.g., other preferences) may be activated, deactivated, associated, disassociated, modified, and/or otherwise defined and/or managed. As shown in the example interface 800, for example, a rule 810-7b regarding “time window(s)” may be activated for various selected parameters 810-7a such as “acceleration monitoring” and/or “location monitoring”. Such activation and/or relation may, in some embodiments, indicate that acceleration monitoring and location monitoring are to be conducted in accordance with one or more “time window(s)” rules 810-7b. Such rules 810-7b may be defined by the customer (e.g., by clicking on and/or otherwise selecting the “time window(s)” text and/or associated selection boxes in the interface 800) and may, for example, define time conditions during which acceleration and/or location should, or should not, be monitored and/or analyzed. In some embodiments, the rule 810-7b may comprise “driving rules” which may, for example, compare local, state, federal, Department of Transportation (DOT), and/or other or customized laws, rules, ordinances, and/or restrictions to associated selected parameters 810-7a, such as to the “speed monitoring” as shown in the example interface 800. In such a manner, for example, monitored speed may be compared to selected and/or defined regulations or restrictions to determine whether a positive or negative driving event has occurred. In some embodiments, “risk zones” rules 810-7b may define geographic areas, road segments, etc., that are associated with various levels of risk (and/or busyness) in which or outside of, for example, location monitoring will be conducted. Similarly, geo-“fencing” rules 810-7b may comprise rules defining geographic boundaries within which and/or outside of which location monitoring is to take place, is not to take place, and/or is to take place in a certain manner (e.g., within or outside of certain zones, such as “privacy” or “personal” zones, location monitoring may be disabled or may still occur, but with a reduced precision or resolution—e.g., instead of utilizing GPS coordinates, postal codes may be monitored).

According to some embodiments, any or all of the various preferences 810 may be associated with an insurance modifier 814. Selections of preferences 810 (and/or combinations of preferences 810) that are less risky and/or otherwise more favorable to an insurer, for example, may result in discounts, lower premiums and/or deductibles, and/or rewards, while more risky (or potentially more risky or involving less oversight) selections may result in surcharges, penalties, and/or higher premiums and/or deductibles. As shown in the example interface 800, for example, the initial data capture window preferences 810-1 being set at “90 days” may result in a ten percent (10%) discount 814-1, the risk zones/geo-“fencing” preferences 810-2 being turned “off” may result in a twenty percent (20%) surcharge 814-2, the monitoring preferences 810-3 being set to enable alerts (e.g., via SMS) may result in a five percent (5%) discount 814-3, the data capture frequency preferences 810-4 being set to five (5) days per month may result in a five percent (5%) discount 814-4, the data capture preferences 810-5 being set to “random” may result in a ten percent (10%) discount 814-5, and/or the forgiveness preferences 810-6 being set to “Gold” level with status alerts enabled may result in a fifteen percent (15%) surcharge 814-6. In some embodiments, the selections made in the matrix of preferences 810-7 may result in a two percent (2%) total discount 814-7, as shown in the example. According to some embodiments, a total modifier 820 may be displayed, showing the total positive or negative (or neutral) effect of application of any or all of the individual insurance modifiers 814 (in the example of FIG. 8, a three percent (3%) surcharge).

According to some embodiments, the interface 800 may comprise a datum selector 840 that may, for example, allow a customer to select from a plurality of available variables for which the interface 800 may be provided. In the example of FIG. 8, the customer may utilize the datum selector 840 to choose between providing and/or showing “premium”, “deductible”, “rewards”, and/or other (“ . . . ”) information. In such a manner, for example, the total modifier 820 may be viewed in context as applied to various variables (e.g., a datum) such as expected and/or current insurance premiums, expected and/or current insurance deductibles, and/or expected and/or current rewards (e.g., credits, airlines miles, and/or other reward points or benefits). In some embodiments, a balance/bank 842 may be provided. The balance/bank 842 may, for example, provide a current and/or expected value associated with any particular variable selected via the datum selector 840. As shown in FIG. 8, for example, the balance/bank may provide the current and/or expected (e.g., quoted) insurance premium level and/or magnitude (with or without the total modifier 820 included; e.g., gross or net).

In some embodiments, buttons or other navigational aids or tools may be provided to allow the customer to switch views and/or go to other data screens, such as the “Go to Grouped Parameter View” button 830 (selection of which, e.g., may cause the interface 700 of FIG. 7 to be provided), the “Go to Itemized Vehicle View” button 834 (selection of which, e.g., may cause the interface 900 of FIG. 9 to be provided), the “Go to Package View” button 836 (selection of which, e.g., may cause the interface 1000 of FIG. 10 to be provided), and/or the “Go to Itemized Parameter View” button 838 (selection of which, e.g., may cause the interface 600 of FIG. 6 to be provided).

FIG. 9 illustrates an exemplary itemized vehicle user interface 900 having a scheduled listing of vehicles 902, along with the itemized parameters 910 selected for each listed vehicle 902. The vehicles may be identified by number, type, year, make, model, VIN, etc. In one embodiment, the user interface 900 shown in FIG. 9 displays the parameters 910 that were selected for each listed vehicle 902 on any or all of the other interfaces 600, 700, 800, 1000, 1100. In another embodiment, a user may select parameters 910 on any or all of the other user interfaces 600, 700, 800, 1000, 1100. In addition to the itemized parameters 910, the estimated premium 924 for each listed vehicle 910 is shown in the right column. A user may add additional vehicles by clicking on (or otherwise selecting) the “ADD ANOTHER VEHICLE” button 904. In some embodiments, buttons or other navigational aids or tools may be provided to allow the customer to switch views and/or go to other data screens, such as the “Go to Grouped Parameter View” button 930 (selection of which, e.g., may cause the interface 700 of FIG. 7 to be provided), the “Go to Preferences View” button 932 (selection of which, e.g., may cause the interface 800 of FIG. 8 to be provided), the “Go to Package View” button 936 (selection of which, e.g., may cause the interface 1000 of FIG. 10 to be provided), and/or the “Go to Itemized Parameter View” button 938 (selection of which, e.g., may cause the interface 600 of FIG. 6 to be provided).

FIG. 10 illustrates an exemplary package view user interface 1000 having a scheduled listing of vehicles 1002, along with a plurality of parameter packages 1010 selected for each listed vehicle 1002. As with the embodiment shown in the interface 900 of FIG. 9, the user interface 1000 of FIG. 10 may display the parameter package 1010 selections made for each listed vehicle 1002 in the other interfaces 600, 700, 800, 900, 1100, in some embodiments, may allow a user to select or update packages of parameters 1010. In addition to the parameter packages 1010, the estimated premium 1024 for each listed vehicle 1002 may be shown in the right column. A user may add additional vehicles by clicking on an “ADD ANOTHER VEHICLE” button 1004. In some embodiments, buttons or other navigational aids or tools may be provided to allow the customer to switch views and/or go to other data screens, such as the “Go to Grouped Parameter View” button 1030 (selection of which, e.g., may cause the interface 700 of FIG. 7 to be provided), the “Go to Preferences View” button 1032 (selection of which, e.g., may cause the interface 800 of FIG. 8 to be provided), the “Go to Itemized Vehicle View” button 1034 (selection of which, e.g., may cause the interface 900 of FIG. 9 to be provided), and/or the “Go to Itemized Parameter View” button 1038 (selection of which, e.g., may cause the interface 600 of FIG. 6 to be provided). In one embodiment, the parameter packages 1010 that are displayed may be based on results of a survey and/or questionnaire.

FIG. 11 illustrates an exemplary user interface 1100 showing various surcharge and discount events. In some embodiments, data descriptive of the events includes the location where the events occurred, e.g., on a map interface as shown. The interface 1100 of FIG. 11 may be displayed, e.g., on the computer screen of a customer or agent accessing the customer's account. Box 1110 identifies select surcharge events and box 1120 identifies select discount events. Box 1130 includes box 1131, which identifies on a map the location where various events occurred, as described in the text of box 1132. Various additional data may also be displayed in FIG. 11. For example, the events in window 1132 may be color coded to indicate the parameters the customer has selected for monitoring.

In an embodiment where a customer allows all data items to be monitored, but only some data items to be utilized, for example, the data items that are utilized may be displayed in green and those not used may be displayed in red. In addition, in some embodiments, the customer may click on the various displayed events using a pointer controlled by a user input device, and the system may calculate and display estimates of what the customer's insurance cost would have been had the customer allowed (or not allowed) data items related to the clicked on events to be used in calculating his premium.

Any or all of the interfaces 600, 700, 800, 900, 1000, 1100 may be altered in various ways. For example, instead of providing fields indicating the maximum possible ranges and predicted ranges of the premium, a pictorial representation of this information may be displayed (e.g., a bar with heavier shading near the estimated value near the center of the bar and lighter at the ends of the bar, representing the outer limits of the premium's likely value).

In some embodiments, the monitored data items may be utilized to reconstruct events, such as automobile accidents. The information from the reconstruction may be utilized, e.g., to affect the payment to third parties and/or subrogation of a claim. For example, the speed of a vehicle colliding with an insured vehicle may be determined based on, e.g., the mass of the two vehicles combined with one or more of the monitored data items (e.g., speed, direction, acceleration, etc.). In another example, the g-force an accident victim was exposed to may be determined based on similar information, and potential fraud may be detected based on medical information indicating that certain types of injuries (e.g., soft tissue damage) are extremely unlikely to occur in certain conditions (e.g., below a certain g-force threshold). Similarly, where geo-“fencing” and/or other location tracking is enabled, various location-based fraud events may be detected based on monitored parameters. When borders outside of agreed-upon travel areas and/or borders into risk zones are crossed, for example, fraud and/or surcharge events may be triggered and/or determined. In some embodiments, location monitoring may be utilized to infer “garaging” locations that, in fraudulent situations, may be indicated (e.g., on policy and/or application papers) in locations that are less risky and/or cheaper to insure than in the location where a vehicle is actually determined to be kept.

The systems, media, and methods described herein may be used for various types of insurance including, for example, automobile, boat, property, worker's compensation, liability, etc. and various combinations of the same. For example, a property owner could agree to have utility usage monitored in order to determine whether the property was vacant, but not for example, monitoring other aspects of home use. A worker's compensation insurance customer (e.g., the insured, the insured representative, a company with insured employees, etc.) might choose to allow payroll monitoring, but not, for example, video monitoring of the worksite.

In some embodiments, data items monitored in relation to one type of activity may be used in determining an insurance premium for various types of policies. For example, a premium for workers compensation insurance may depend on monitoring data items of a worker's driving. A customer may select the parameters to be monitored for respective policies (or, e.g., for his employees policies). For example, a customer may decide to allow her driving speed to be used in determining her automobile insurance premium, but not for her workers compensation premium. In another example, a customer's monitored data items may be used to generate a score, e.g., representative of the customer's risk level. This level may be indicative of the customer's risk across various areas and may be used as a factor in determining a premium for various types of policies (e.g., home owner's, workers compensation, etc.). Selections of monitored parameters for given policies may also be made at the fleet level. For example, the premium for workers compensation insurance for drivers associated with a fleet may depend on the monitored data items associated with the fleet.

As described herein, an insurance company may allow a customer to select various parameters and/or preferences. In some embodiments, parameters and/or preferences may be set, suggested, and/or required by the insurance company (or a third-party). The parameters and/or preferences available for selection and/or required for monitoring may depend on various considerations, including, e.g., one or more of: a type of risk (e.g., based on whether the customer is a wholesaler, a contractor, and manufacturer, etc); a product or collection of products (e.g., which insurance product or line of products is at issue); a risk assessment and/or classification of the customer (e.g., non-standard/risky customers may be required to have more parameters monitored than a standard/lower risk customer—such as, e.g., a non-standard customer may be required to have video monitoring); history based (e.g., an individual or collective driving history, such as, e.g., requiring all drivers with more than five points to be monitored with an extensive set of data items); loss history (e.g., drivers with more than a certain number of claims or total a total claim over a certain dollar value may be required to have video monitoring); or other types of insurance related considerations.

Some embodiments may combine various data items to calculate an indication of certain risks. For example, some embodiments may combine data related to hard breaking and swerving to calculate that a near miss has occurred. The calculated indication may be used as indication of risk on which insurance cost may be based. For example, if a driver has one near miss every four (4) years, the insurance company may consider those near misses not to be an indication that the driver is any more likely to get in an accident that a driver with no near misses. However, the insurance company may consider a customer that has six (6) near misses in one (1) year to be very likely to get in an accident soon, and decide to adjust that driver's insurance premium upward according to the driver's high number of near misses. In addition, the insurance company may impose higher premiums on any driver that does not elect a near miss package or otherwise select data items and/or monitoring preferences that allow the insurance company to determine the occurrence of near misses.

Various embodiments described herein enable insurance companies to aid customers in both identifying hazards and establishing associated controls to reduce, limit, eliminate, and/or manage those hazards. Any situation that could cause an insured to experience loss is a potential hazard. Not all hazards are covered by insurance. Customers may obtain or purchase risk control or risk management services from an insurance company with or without purchasing other insurance products. The insurance company may help customers to identify hazards through many methods including providing educational materials, classes, etc., performing inspections, recommending organizational structures, policies, operational methods, etc., which help to identify potential hazards. Once customers have identified a potential hazard, the insurance company may further assist by providing educational materials, classes, etc.; recommending organizational structures, policies, operational methods, etc. for reducing, limiting, eliminating or controlling those hazards.

Some situations are more hazardous than others, and require varying kinds of risk management strategies and applications. By choosing to monitor certain items as described herein, both the identification of hazards and the associated controls may be improved. Also, by choosing to monitor certain items, the customer may consequently be able to utilize certain of the insurance company's risk control or risk management service or product offerings which depend upon that particular item being monitored (and/or method or type of monitoring), and so would otherwise not be available to that customer. In some embodiments, e.g., hazards may be measure by monitoring data items and controlled by adjusting a premium.

It will be readily apparent that the various methods and algorithms described herein may be implemented by, e.g., appropriately programmed general purpose computers and computing devices. Typically a processor (e.g., one or more microprocessors) will receive instructions from a memory or like device, and execute those instructions, thereby performing one or more processes defined by those instructions. Further, programs that implement such methods and algorithms may be stored and transmitted using a variety of media (e.g., computer-readable media) in a number of manners.

In some embodiments, hard-wired circuitry or custom hardware may be used in place of, or in combination with, software instructions for implementation of the processes of various embodiments. Thus, embodiments are not limited to any specific combination of hardware and software. Accordingly, a description of a process likewise describes at least one apparatus for performing the process, and likewise describes at least one computer-readable medium and/or memory for performing the process. The apparatus that performs the process can include components and devices (e.g., a processor, input and output devices) appropriate to perform the process. A computer-readable medium can store program elements appropriate to perform the method.

It is to be understood that the embodiments described above are not limited in its application to the details of construction and to the arrangements of the components set forth in the above description or illustrated in the drawings. The invention is capable of other embodiments and of being practiced and carried out in various ways. Also, it is to be understood that the phraseology and terminology employed herein are for the purpose of description and should not be regarded as limiting.

For example, the specific sequence of described process may be altered so that certain processes are conducted in parallel or independent with other processes, to the extent that the processes are not dependent upon each other. Thus, the specific order of steps described herein are not to be considered implying a specific sequence of steps to perform the above described process. Other alterations or modifications of the above processes are also contemplated. Accordingly, those skilled in the art will appreciate that the conception upon which this disclosure is based may readily be utilized as a basis for the designing of other structures, methods, and systems for carrying out the several purposes of the present invention. It is important, therefore, that the invention be regarded as including equivalent constructions to those described herein insofar as they do not depart from the scope of the present invention.

The many features and advantages of the invention are apparent from the detailed specification, and thus, it is intended by the appended claims to cover all such features and advantages of the invention which fall within the scope of the invention. Further, since numerous modifications and variations will readily occur to those skilled in the art, it is not desired to limit the invention to the exact construction and operation illustrated and described, and accordingly, all suitable modifications and equivalents may be resorted to, falling within the scope of the invention, as defined by the claims.

What is claimed is:

1. A vehicle monitoring system for accepting as input via a Graphical User Interface (GUI), rules defined by user selections received via a plurality of selectable interface options, the rules governing how data is to be recorded by a telematics device removably coupled to a vehicle, the system comprising:
the telematics device, comprising an accelerometer and a wireless communication interface in communication, via a remote telecommunications device, with a communications device of a controller device, the telematics device being operatively coupled to the vehicle to measure a set of parameters and corresponding data descriptive of an operation of the vehicle, the set of parameters comprising at least two parameters from a plurality of types of parameters;
the controller device, comprising a plurality of microprocessors, and the communications device comprising a communications router in communication via the remote telecommunications device with the telematics device; and
a non-transitory computer readable memory in communication with the controller device, the non-transitory computer readable memory storing (i) telematics data monitoring program instructions and (ii) the rules defined by the user selections received via the plurality of selectable interface options, the instructions and rules, when executed by the plurality of microprocessors, resulting in:
generating the GUI, the GUI comprising the plurality of selectable interface options, each interface option being assigned to one of:
(i) a particular parameter from the set of parameters measurable by the telematics device; and
(ii) a particular rule for monitoring at least one parameter from the set of parameters measurable by the telematics device, the particular rule defining at least one of: (1) a time window for monitoring; (2) a frequency for monitoring; and (3) a geographic-based monitoring rule;
transmitting, by the communications device and to a remote device associated with the user, instructions defining the generated GUI, the instructions being operable to cause the GUI to be output via a display device of the remote device associated with the user;
receiving, in response to the transmitting and by the communications device, an indication of a user selection of a subset of the selectable interface options, the indication of the user selection defining the rules governing how data is to be recorded by the telematics device;
transmitting, in response to the receiving of the indication of the user selection, by the controller device and to the telematics device, an instruction to record a subset of the data in accordance with the rules governing how data is to be recorded by the telematics device;
recording, by the telematics device, the subset of the data;
transmitting, by the wireless communication interface of the telematics device, via the remote telecommunications device, and to the communications device of the controller device, the recorded subset of the data;
receiving, by the communication device of the controller device, and from the telematics device, the subset of the data, the subset of the data complying with the rules governing how data is to be recorded by the telematics device;
determining, by the controller device, a base value indicator for a telematics data monitoring program;
determining, by the controller device and based on one or more of: (i) the user selection defining the rules governing how data is to be recorded by the telematics device, and (ii) the subset of the data complying with the rules governing how data is to be recorded by the telematics device, a value modifier;
calculating, by the controller device and based on a combination of the base value indicator and the value modifier, an adjusted value indicator for the telematics data monitoring program; and
outputting, by the controller device, a graphical indication of the adjusted value indicator for the telematics data monitoring program.

2. The system of claim 1, wherein the plurality of types of parameters comprises:
at least one driver parameter type, comprising at least one of: (i) a driver heart rate parameter, (ii) a driver respiration rate parameter, and (iii) a driver head position parameter;
at least one environmental parameter type, comprising at least one of: (i) a temperature parameter; (ii) a weather condition parameter (iii) a road condition parameter; and (iv) a traffic condition parameter; and
at least one vehicle parameter type, comprising at least one of: (i) a speed parameter; (ii) an acceleration parameter; (iii) a location parameter; (iv) an orientation parameter; (v) a weight parameter; (vi) a fuel quantity parameter; (vii) a tire pressure parameter; and (viii) an engine or motor parameter.

3. The system of claim 1, wherein the indication of the user selection defining the rules governing how data is to be recorded by the telematics device comprises a definition of the time window for monitoring, and wherein the time window for monitoring comprises at least one of: (i) one or more certain days of the week; (ii) one or more certain times of the day; and (iii) one or more certain seasons.

4. The system of claim 3, wherein the one or more certain days of the week comprise at least one of: (i) defined workdays; and (ii) defined weekend days.

5. The system of claim 3, wherein the one or more certain times of the day comprise at least one of: (i) defined work hours; (ii) defined commuting hours; (iii) daylight hours; and (iv) nighttime hours.

6. The system of claim 3, wherein the one or more certain seasons comprises a defined winter driving season.

7. The system of claim 1, wherein the indication of the user selection defining the rules governing how data is to be recorded by the telematics device comprises a definition of the frequency for monitoring.

8. The system of claim 1, wherein the indication of the user selection defining the rules governing how data is to be recorded by the telematics device comprises a definition of the geographic-based monitoring rule, and wherein the geographic-based monitoring rule comprises at least one of: (i) a particular driver being located in a particular area, zone, road, or structure; and (ii) a particular vehicle being located in the particular area, zone, road, or structure.

9. The system of claim 1, wherein the particular rule for monitoring at least one parameter from the set of parameters measurable by the telematics device defines an environmental condition for monitoring.

10. The system of claim 9, wherein the indication of the user selection defining the rules governing how data is to be recorded by the telematics device comprises a definition of the environmental condition for monitoring, and wherein the environmental condition for monitoring comprises at least one of: (i) an occurrence of a particular road condition; and (ii) an occurrence of a particular weather condition.

11. The system of claim 1, wherein the particular rule for monitoring at least one parameter from the set of parameters measurable by the telematics device defines legal or regulatory condition for monitoring.

12. The system of claim 11, wherein the indication of the user selection defining the rules governing how data is to be recorded by the telematics device comprises a definition of the legal or regulatory condition for monitoring, and wherein the legal or regulatory condition for monitoring comprises at least one of: (i) a monitoring rule based on a teen driving law; and (ii) a monitoring rule based on a roadway vehicle type restriction.

13. The system of claim 1, wherein the remote device associated with the user comprises the telematics device.
